(19) United States
(12) Patent Application Publication
(10) Pub. No.: US 2018/0250341 A1
(43) Pub. Date: Sep. 6, 2018

(54) METHOD OF REPAIRING DAMAGED BLOOD-SPINAL CORD BARRIER (BSCB)

(71) Applicants: Svitlana Garbuzova-Davis, Tampa, FL (US); Paul Ronald Sanberg, Tampa, FL (US)

(72) Inventors: Svitlana Garbuzova-Davis, Tampa, FL (US); Paul Ronald Sanberg, Tampa, FL (US)

(73) Assignee: UNIVERSITY OF SOUTH FLORIDA, Tampa, FL (US)

(21) Appl. No.: 15/905,522

(22) Filed: Feb. 26, 2018

Related U.S. Application Data
(60) Provisional application No. 62/463,396, filed on Feb. 24, 2017.
(60) Provisional application No. 62/617,002, filed on Jan. 12, 2018.

(51) Int. Cl. A61K 35/28; A61P 25/28; A61K 9/00

(52) U.S. Cl. CPC A61K 35/28; A61K 2035/124; A61K 9/0019; A61P 25/28

(57) ABSTRACT

The disclosure provides methods of administering a composition comprising human bone marrow CD34+ (hBM34+) cells to a mammal in need thereof, whereby vascular damage in the blood-central nervous system barrier (B-CNS-B), such as in the blood-spinal cord barrier, of the mammal is repaired. The disclosure also provides a method of treating amyotrophic lateral sclerosis (ALS) in a mammal by administering a composition comprising human bone marrow CD34+ cells (hBM34+).

### CROSS REFERENCE TO RELATED APPLICATIONS

This application claims priority to U.S. Provisional Application No. 62/463,396, filed Feb. 24, 2017, and U.S. Provisional Application No. 62/617,002, filed Jan. 12, 2018, which are incorporated herein by reference in their entirety.

### GOVERNMENT FUNDING

The subject matter of this invention was made with Government support under Federal Grant No. R01 NS090962 awarded by the National Institutes of Health. The Government has certain rights in this invention.

### TECHNICAL FIELD

The present disclosure relates to methods of repairing vascular damage in the blood-central nervous system barrier, such as the blood-spinal cord barrier.

### BACKGROUND

Amyotrophic lateral sclerosis (ALS) is a rapidly progressing debilitative neurodegenerative disorder characterized by motor neuron degeneration in the brain and spinal cord, leading to paralysis and eventual death within 3-5 years after symptom onset. The majority of ALS cases (90-95%) are sporadic (SALS) with unknown cause. Approximately 5-10% of cases are genetically linked (familial cases, FALS), of which 20% have a missense mutation in the Cu/Zn superoxide dismutase 1 (SOD1) gene. Additional mutations in the transactive response DNA binding protein (TARDBP; TDP-43), fused in sarcoma/translocated in liposarcoma (FUS/TLS), angiogenin (ANG), and chromosome 9 open reading frame 72 (C90RF72) genes have been identified in FALS cases, and some of these mutations were noted in SALS cases. Despite the genetic variants, SALS and FALS share clinical and pathological presentations. The treatment options for ALS are mostly supportive. The only approved drugs for ALS by the United States of America Federal Drug Administration are riluzole (marketed as RILUTEK® and TEGLUTIK®) and the recently approved RADICAVA® (edaravone).

ALS is a multifactorial disease with numerous effectors underlying disease pathogenesis such as glutamate excitotoxicity, oxidative stress, mitochondrial dysfunction, impaired axonal transport, aberrant RNA metabolism, protein aggregations, dysfunctional autophagy, modified glial cell function, altered neurotrophic factor levels, immune reactivity, and neuroinflammation. Accumulating evidence has also shown breakdown of the blood-central nervous system-barrier (B-CNS-B), i.e. the blood-brain barrier (BBB) and the blood-spinal cord barrier (BSCB), potentially representing an additional pathogenic mechanism identifying ALS as a neurovascular disease. The essential role of the B-CNS-B is to maintain homeostasis within the CNS by preventing diffusion of detrimental factors from the blood circulation to the CNS. The barriers are composed of endothelial cells and tight junctions that interact with pericytes, astrocytes, perivascular macrophages, and the basal lamina to form an integrated microvascular unit. B-CNS-B impairment has been demonstrated in ALS patients and the G93A SOD1 mouse model of ALS. In the G93A mice, endothelial cell degeneration and astrocyte end-feet alterations have been observed before disease onset as well as at different stages of the disease. Importantly, BSCB alterations were indicated in SOD1 mutant mice and rats prior to motor neuron degeneration and neuroinflammation, suggesting vascular damage as an early ALS pathological event. Moreover, compromised BSCB integrity was demonstrated by Evans blue dye extravasation into CNS parenchyma in pre-symptomatic and symptomatic G93A rodents. Reductions of tight junction proteins such as zonula occludens 1 (ZO-1), occludin, and claudin-5 have also been detected in the ventral horn of the lumbar spinal cord in G93A SOD1 mice at pre-symptomatic and symptomatic disease stages. However, decreased levels of tight junction proteins were determined in G93A SOD1 rats mainly at the symptomatic stages. Studies using post-mortem human ALS tissues in several laboratories also support disease-related BSCB dysfunction by demonstrating endothelial cell degeneration, astrocyte end-feet alterations, and reduction of tight junction protein expressions. Thus, it is possible that the initiating pathological trigger for ALS is a dysfunctional B-CNS-B, allowing detrimental factors from the systemic circulation to penetrate the CNS and initiate inflammation fostering motor neuron degeneration.

Since B-CNS-B dysfunction, such as damage to the BSCB, may be a potential contributor to ALS pathogenesis, there remains a need for agents and methods for repairing damage to the B-CNS-B as a treatment for ALS.

### SUMMARY OF THE INVENTION

The disclosure provides a method of repairing vascular damage in the blood-central nervous system (CNS) barrier (B-CNS-B) of a subject. The method includes administering a composition that includes human bone marrow CD34+ (hBM34+) cells to a subject in need thereof, whereby the vascular damage in the B-CNS-B of the subject is repaired.

The disclosure also provides a method of treating amyotrophic lateral sclerosis (ALS) in a subject. The method includes administering a composition that includes human bone marrow CD34+ cells (hBM34+) to a subject suffering from ALS, whereby ALS is treated in the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show the distribution of microhemorrhages in the cervical (FIG. 1A) and lumbar (FIG. 1B) spinal cords of G93A ALS symptomatic control mice, media-injected mice, and mice treated with low, mid, or high hBM34+ cell doses at 17 weeks of age and histologically stained with Perls' Prussian blue. Quantitative analysis of the distribution of microhemorrhages in the cervical (FIG. 1C) and lumbar (FIG. 1D) spinal cords. *p<0.05., **p<0.01. FIG. 1E is a schematic depiction of microhemorrhage distribution within the C4-C6 (left) and L3-L5 (right) segments of the spinal cord of G93A ALS symptomatic mice.

FIGS. 2A-2I show the presence of endothelial cells in the spinal cords G93A ALS symptomatic mice treated with different doses of hBM34+ cells as determined by immunohistochemical staining for human Von Willebrand Factor (vWF). Immunopositive vWF expression (FIG. 2, arrowheads) was seen in cells of the cervical (FIGS. 2A,2A′) and lumbar (FIGS. 2E,2E′) spinal cords of mice receiving the low cell dose, in the cervical (FIGS. 2B,2B′) and lumbar (FIGS. 2F,2F′) spinal cords of mice with mid cell dose, and in numerous capillaries in the cervical (FIGS. 2C,2C′) and lumbar (FIG. 2G) spinal cords of mice treated with high cell dose. No immunoexpression of vWF was found in the spinal cords of media-injected mice (FIGS. 2D,2H). Dose-dependent quantitative analysis of fluorescent vWF expression in ventral gray matter of the cervical and lumbar spinal cords is shown in FIG. 2I. *p<0.05, **p<0.01, ***p<0.001.

FIGS. 3A-3R show the immunohistochemical staining for HuNu and CD45 in the cervical and lumbar spinal cords of G93A ALS symptomatic mice receiving low (FIGS. 3A-3C″ and FIGS. 3J-3L″, respectively), mid (FIGS. 3D-3E″, FIGS. 3M-3N, respectively), and high (FIGS. 3F-3H″, FIGS. 3O-3Q″, respectively) hBM34+ cell doses. No cells expressing HuNu or CD45 were determined in the cervical (FIGS. 3I-3I″) or lumbar (FIGS. 3R-3R″) spinal cord from media-injected mice. Merged images were costained with DAPI.

FIGS. 4A-4E show the effect of different doses of hBM34+ cells on the disease markers in G93A ALS symptomatic mice. Mice were monitored for body weight (FIG. 4A), hindlimb extension reflex (FIG. 4B), muscle strength (FIG. 4C), and length of time maintained on a rotarod (FIG. 4D) before and after treatment with hBM34+ cells. Percentages of mice remaining on rotarod for at least 180 seconds were quantified starting at fourteen weeks of age for each dosage group (FIG. 4E). *p<0.05, **p<0.01.

FIGS. 5A-5F show characteristics of protoplasmic astrocytes in the cervical spinal cord of G93A mice. Immunohistochemical staining of astrocytes with GFAP antibody was completed in G93A ALS symptomatic control mice (FIG. 5A), media-injected mice (FIG. 5B), and mice treated with low (FIG. 5C), mid (FIG. 5D), or high (FIG. 5E) hBM34+ cell doses at 17 weeks of age. Quantitative analysis of GFAP immunoexpression is shown in FIG. 5F. ***p<0.001.

FIGS. 6A-6E show characteristics of protoplasmic astrocytes in the lumbar spinal cord of G93A mice. Immunohistochemical staining of astrocytes with GFAP antibody was completed in G93A ALS symptomatic control mice (FIG. 6A), media-injected mice (FIG. 6B), and mice treated with low (FIG. 6C), mid (FIG. 6D), or high (FIG. 6E) hBM34+ cell doses at 17 weeks of age. Quantitative analysis of GFAP immunoexpression is shown in FIG. 6F. *p<0.05, ***p<0.001.

FIGS. 7A-7J show characteristics of perivascular astrocytes in the cervical and lumbar spinal cord of G93A ALS symptomatic mice. FIG. 7A shows the immunohistochemical staining of perivascular astrocytes with GFAP antibody in the cervical (FIGS. 7A-7E′) and lumbar (FIGS. 7F-7J′) spinal cords of control mice (FIGS. 7A-7A′, FIGS. 7F-7F′), media-injected mice (FIGS. 7B-7B′, FIGS. 7G-7G′), and mice treated with low (FIGS. 7C-7C′, FIGS. 7H-7H′), mid (FIGS. 7D-7D′, FIGS. 7I-7I′), or high (FIGS. 7E-7E′, FIGS. 7J-7J′) hBM34+ cell doses. Quantitative analysis of GFAP immunoexpression is shown in FIG. 7K. **p<0.01, ***p<0.001.

FIGS. 8A-8J show the characteristics of microglial cells in the ventral horn of spinal cords from G93A ALS symptomatic mice. Immunohistochemical staining of microglia using anti-Iba-1 antibody was performed on the cervical (FIGS. 8A-E) and lumbar (FIGS. 8F-J) spinal cord sections of control mice, media-injected mice, and mice treated with low, mid, or high hBM34+ cell doses at 17 weeks of age.

FIGS. 9A-9L show the characteristics of motor neurons in the cervical and lumbar spinal cords (FIGS. 9A-J) of G93A ALS symptomatic control mice, media-injected mice, and mice treated with low, mid, or high hBM34+ cell doses at 17 weeks of age. Sections were histologically stained with cresyl violet and stereological motor neuron counts levels were quantified in the cervical (FIG. 9K) and lumbar (FIG. 9L) spinal cords. **p<0.01, ***p<0.001.

### DETAILED DESCRIPTION

The disclosure is predicated, at least in part, on the discovery that transplantation of unmodified human bone marrow cells into symptomatic ALS mice leads to restoration of capillary integrity in the spinal cord as determined by detection of microhemorrhages, as well as endothelial cell differentiation, capillary cell engraftment, re-established perivascular end-feet astroycytes, and improved motor neuron function. The transplanted hBM34⁺ cells differentiated into endothelial cells in the spinal cords of all cell-treated animals. Immunopositive vWF expression were observed within capillaries of cervical and lumbar spinal cords of low dose mice. Adherent cells form distinctive lining in capillary walls of cervical and lumbar spinal cords of mid dose mice. In mice receiving the high cell dose, cells engrafted into numerous microvessel walls of the cervical and lumbar spinal cord, showing significantly increased vWF immunoexpression. The endothelial cell differentiation and capillary engraftment of transplanted hematopoietic stem cells indicated reparative processes towards BSCB restoration that can be dosage dependent. The significant decrease in spinal cord microhemorrhages of symptomatic ALS mice treated with the highest cell dose is also concomitant with microvascular repair. Repair of the BSCB in ALS may be involved for increased motor neuron survival. Cell therapy for restoration of this barrier is a promising therapeutic strategy for ALS. The disclosure provides translational outcomes supporting the utility of hBM34⁻ cell transplantation at optimal dose as a therapeutic strategy leading to potential BSCB restoration in ALS patients.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are hereby incorporated by reference in their entirety. The materials, methods, and examples disclosed herein are illustrative only and not intended to be limiting.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“About” is used synonymously herein with the term “approximately.” Illustratively, the use of the term “about” indicates that values slightly outside the cited values, namely, plus or minus 10%. Such values are thus encompassed by the scope of the claims reciting the terms “about” and “approximately.”

The use of the terms “a” and “an” and “the” and “at least one” and similar referents in the context of describing the invention (especially in the context of the following claims) are to be construed to cover both the singular and the plural, unless otherwise indicated herein or clearly contradicted by context.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

“Amyotrophic lateral sclerosis” and “ALS” as used interchangely herein refers to a group of rare neurological diseases that mainly involve the nerve cells (neurons) responsible for controlling voluntary muscle movement, like chewing, walking, and talking. ALS is a progressive disease in which the symptoms get worse over time. ALS is a motor neuron disease that is caused by gradual deterioration (degeneration) and death of motor neurons, which are nerve cells that extend from the brain to the spinal cord and to muscles throughout the body. These motor neurons initiate and provide vital communication links between the brain and the voluntary muscles.

“Blood-central nervous system barrier,” “blood-CNS barrier,” and “B-CNS-B” as used interchangeably herein refers to the physical barrier between blood and the CNS. The blood-CNS includes the blood-brain barrier, the blood-spinal cord barrier and the blood-cerbrospinal fluid (CSF) barrier and protects the CNS from both toxic and pathogenic agents in the blood. Disruption of the blood-CNS barrier plays a key role in a number of CNS disorders, particularly those associated with neurodegeneration.

“Blood-spinal cord barrier” and “BSCB” as used interchangeably herein refers to the barrier between blood and the spinal cord. The BSCB maintains the fluid microenvironment of the spinal cord.

The term “repair” as used herein refers to the correction, reversal, improvement, alleviation, or restoration, in whole or in part, of something that is damaged, injured, or defective.

“Subject” and “patient” as used herein interchangeably refers to any vertebrate, including, but is not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject can be a human or a non-human. The subject or patient can be undergoing other forms of treatment. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Suitable subjects include both males and females and subjects of any age, including embryonic (e.g., in utero or in ovo), infant, juvenile, adolescent, adult and geriatric subjects. In some embodiments, a subject of this disclosure is a human.

A “subject in need” of the methods of the disclosure can be a subject known to have, suspected of having, or having an increased risk of developing an neurological-disease or condition, such as ALS.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to a disease or a condition—such as ALS the term refers to, e.g., a decrease in the symptoms or other manifestations of the disease or condition, such as ALS. In some embodiments, treatment provides a reduction in symptoms or other manifestations of the disease or condition, such as ALS by at least about 5%, e.g., about 10%, 15%, 20%, 25%, 30%, 35%, 40%, 45%, 50% or more.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### 2. METHODS OF REPAIRING VASCULAR DAMAGE IN THE BLOOD-CENTRAL NERVOUS SYSTEM (CNS) BARRIER (B-CNS-B)

The disclosure provides methods of repairing vascular damage in the blood-central nervous system (CNS) barrier (B-CNS-B) of a subject. The method includes administering a composition that includes human bone marrow CD34+ (hBM34+) cells to a subject in need thereof. In the context of the disclosed method, the vascular damage can be located in the B-CNS-B, and in some embodiments, the vascular damage can be located in the BSCB. The disclosed method can be used to repair any type of vascular damage, injury, or trauma to any type of blood vessel or blood vessel network located in the B-CNS-B or BSCB, or particular vascular damage that contributes to the development of ALS. In some embodiments, the vascular damage can include endothelial cell degeneration, astrocyte end-feet alteration, tight junction protein downregulation, capillary permeability, capillary rupture, or combinations thereof. For example, in one embodiment, the vascular damage may include alteration of astrocyte end-feet, which ensheathe blood vessels in the brain and can provide structural integrity to the cerebral vasculature. In another embodiment, vascular damage can include downregulated or dysfunctional tight junction proteins, which are proteins that form the tight junction (TJ) barrier between closely associated areas of two cells whose membranes join together forming a barrier virtually impermeable to fluid. In other embodiments, vascular damage includes endothelial cell degeneration, such as degeneration of microvessels in the brain and spinal cord. Vascular damage may also include compromised capillary integrity in the spinal cord or BSCB. Capillary integrity is “compromised” when capillaries exhibit permeability or ruptures within a particular capillary segment. “Microhemorrhages” within the CNS parenchyma are indicative of capillary rupture within the B-CNS-B, and these capillary ruptures may be identified through detection of ferric iron deposits derived from blood compartment.

The methods described herein may be used to repair any suitable type of vascular damage to the B-CNS-B, such that the function or activity of the B-CNS-B is restored in whole or in part. In other words, any degree of correction, improvement, alleviation, or restoration of a particular vascular damage or injury is considered a “repair” of that damage in the context of this disclosure. In some embodiments, the repair of vascular damage can include endothelial cell differentiation, capillary cell engraftment, reduced capillary permeability or capillary ruptures as compared to a subject not administered the composition, re-establishment of perivascular end-feet astrocytes, or combinations thereof. For example, the repair of vascular damage may include replacement of endothelial cells such that the level of microvessels in the brain and spinal cord is increased. In another embodiment, repair of vascular damage can include capillary cell engraftment. In other embodiments, repair of vascular damage may include reduction of capillary permeability or capillary ruptures often accompanied by increased in expression of protein that form the tight junction barrier between cells. In some embodiments, the repair of vascular damage can include a reduction in capillary ruptures as compared to a subject not administered the composition that includes hBM34+ cells.

In some embodiments, the administration of the composition that includes hBM34+ cells improves motor neuron survival as compared to a subject not administered the composition. In some embodiments, the administration of the composition that includes hBM34+ cells improves behavioral symptoms as compared to a subject not administered the composition.

### 3. METHOD OF TREATING AMYOTROPHIC LATERAL SCLEROSIS (ALS) IN A SUBJECT

The disclosure provides methods of treating amyotrophic lateral sclerosis (ALS) in a subject. The method includes administering a composition that includes human bone marrow CD34+ cells (hBM34+) to a subject suffering from ALS thereby treating the ALS in the subject. In some embodiments, the administration of the composition that includes hBM34+ cells induces repair of vascular damage in the blood-spinal cord barrier (BSCB) of the subject.

s4. hBM34+ Cells

The disclosed methods utilize human bone marrow CD34+ (hBM34+) cells. Stem cells are known to secrete various growth or trophic factors that may modulate the local microenvironment to rescue diseased motor neurons. Bone marrow is a primary source of the putative endothelial progenitor cells (EPCs). These progenitor cells can be derived from hematopoietic stem cells or cells of endothelial lineage. EPCs can be enriched in CD34+/CD45− cell populations and are not derived from CD133+ or CD45+ cells. CD34+ cells are pluripotent hematopoietic stem cells, capable of long-term in vitro self-renewal and of differentiation into multiple hematopoietic cell lineages that fully repopulate blood cells throughout adulthood. However, lineage potential of the hematopoietic progenitors during proliferation, commitment to multipotential differentiation, and maturation are controlled by various intrinsic properties and microenvironmental factors. Since EPCs can be derived from CD34+ cells, human bone marrow CD34+ (hBM34+) cells can be a cell source for improvement of motor neuron survival, and by extension, behavioral symptoms, in ALS and for B-CNS-B restoration in ALS.

In one embodiment of the disclosure administration of a composition that includes hBM34+ cells improves motor neuron survival as compared to a subject not administered the composition. In an alternate embodiment, administration of a composition that includes hBM34+ cells improves behavioral symptoms as compared to a subject not administered the composition.

### 5. COMPOSITIONS, PHARMACEUTICAL COMPOSITIONS, AND FORMULATIONS

Embodiments of the present disclosure also provide compositions, pharmaceutical compositions, and formulations that include hBM34+ cells. The disclosed compositions, pharmaceutical compositions, and formulations can be used to repair vascular damage in the B-CNS-B, such as in the BSCB. The disclosed compositions, pharmaceutical compositions, and formulations can include hBM34+ cells, in amounts that may be particularly effectively in treating male subjects and/or female subjects. In some embodiments, the compositions, pharmaceutical compositions, and formulations can include hBM34+ cells in a particular formulation that may be more effective in treating male subjects compared to female subjects. In some embodiments, the compositions, pharmaceutical compositions, and formulations can include hBM34+ cells in a particular formulation that may be more effective in treating female subjects compared to male subjects. In some embodiments, the compositions, pharmaceutical compositions, and formulations can include hBM34+ cells in a particular formulation that may have the same effectiveness in treating female subjects compared to male subjects.

The compositions, pharmaceutical compositions, and formulations may include a “therapeutically effective amount” or a “prophylactically effective amount” of hBM34+ cells. A “therapeutically effective amount” refers to an amount effective, at dosages and for periods of time necessary, to achieve the desired therapeutic result. A therapeutically effective amount of the compositions may be determined by a person skilled in the art and may vary according to factors such as the disease state, age, sex, and weight of the individual, and the ability of the compositions to elicit a desired response in the individual. A therapeutically effective amount is also one in which any toxic or detrimental effects of hBM34+ cells are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of hBM34+ cells calculated to produce the desired therapeutic effect in association with the required pharmaceutical carrier. The specification for the dosage unit forms are dictated by and directly dependent on (a) the unique characteristics of hBM34+ cells, and the particular therapeutic or prophylactic effect to be achieved, and (b) the limitations inherent in the art of compounding such composition that modulates hBM34+ cells, for the treatment of sensitivity in individuals.

It is to be noted that dosage values may vary with the type and severity of the condition to be alleviated. Further, hBM34+ cells dose may be determined by a person skilled in the art and may vary according to factors such as the disease state, age, sex, and weight of the individual, and the ability of hBM34+ cells to elicit a desired response in the individual. The dose is also one in which toxic or detrimental effects, if any, of hBM34+ cells are outweighed by the therapeutically beneficial effects. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. In some embodiments, the dosage can include at least about 1×10⁴to at least about 5×10⁶ of hBM34+ cells. In some embodiments, the dosage can include at least about 1×10⁴to at least about 5×10⁶, at least about 5×10⁴to at least about 5×10⁶, at least about 10×10⁴to at least about 5×10⁶, at least about 50×10⁴to at least about 5×10⁶, at least about 100×10⁴ to at least about 5×10⁶, 1×10⁴ to at least about 1×10⁶, at least about 5×10⁴to at least about 1×10⁶, at least about 10×10⁴to at least about 1×10⁶, or at least about 50×10⁴to at least about 1×10⁶ of hBM34+ cells. In some embodiments, the dosage can include at least about 1×10⁴, at least about 5×10⁴, at least about 1×10⁵, at least about 5×10⁵, at least about 1×10⁶, or at least about 5×10⁶ of hBM34+ cells. In some embodiments, the dosage can include at least about 5×10⁴, at least about 5×10⁵, or at least about 1×10⁶ of hBM34+ cells.

The compositions, pharmaceutical compositions, and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Various delivery systems are known and can be used to administer one or more of hBM34+ cells, and a prophylactic agent or therapeutic agent useful for preventing, managing, treating, or ameliorating ALS, or one or more symptoms thereof, e.g., encapsulation in liposomes, microparticles, microcapsules. Methods of administering a prophylactic or therapeutic agent of the invention include, but are not limited to, parenteral administration (e.g., intradermal, intramuscular, intraperitoneal, intravenous and subcutaneous), epidurala administration, intratumoral administration, and mucosal administration (e.g., intranasal and oral routes). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

If the pharmaceutical composition is administered orally, the pharmaceutical compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The pharmaceutical compositions may be administered by and formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use. The methods of the invention can additionally include of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The pharmaceutical compositions may be formulated as neutral or salt forms. Pharmaceutically acceptable salts include those formed with anions such as those derived from hydrochloric, phosphoric, acetic, oxalic, tartaric acid, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, compositions can be dispensed with an infusion bottle containing sterile pharmaceutical grade water or saline. Where the mode of administration is by injection, an ampoule of sterile water for injection or saline can be provided so that the ingredients may be mixed prior to administration.

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application.

In certain embodiments, hBM34+ cells, may be orally administered, for example, with an inert diluent or an assimilable edible carrier. HBM34+ cells (and other ingredients, if desired) may also be enclosed in a hard or soft shell gelatin capsule, compressed into tablets, or incorporated directly into the subject's diet. For oral therapeutic administration, hBM34+ cells may be incorporated with excipients and used in the form of ingestible tablets, buccal tablets, troches, capsules, elixirs, suspensions, syrups, wafers, and the like. To administer hBM34+ cells by other than parenteral administration, it may be necessary to coat hBM34+ cells with, or co-administer hBM34+ cells with, a material to prevent its inactivation.

hBM34+ cells, can be used alone or in combination to treat ALS, or any other disease associated with the B-CNS-B. It should further be understood that the combinations are those combinations useful for their intended purpose.

### 6. ROUTES OF ADMINISTRATION

Provided herein is a method for delivering the pharmaceutical formulations, preferably compositions described above, for providing hBM34+ cells. The compositions can be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition can be administered as a suitably acceptable formulation in accordance with normal veterinary practice. The veterinarian can readily determine the dosing regimen and route of administration that is most appropriate for a particular animal. The compositions can be administered by traditional syringes or needleless injection devices.

In some embodiments, the composition that includes hBM34+ cells is administered intravenously. In some embodiments, the composition that includes hBM34+ cells is administered to the jugular vein. In some embodiments, the composition that includes hBM34+ cells is administered to the mammal once during a therapeutic period. In some embodiments, the composition that includes hBM34+ cells is administered to the mammal two or more times during a therapeutic period.

### 7. KITS

Provided herein is a kit, which can be used to repairing vascular damage. The kit includes compositions, pharmaceutical compositions, or formulations that include hBM34+ cells, as described above, and instructions for using said compositions. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions. In some embodiments, the kits further can include buffers, reagents, and the like.

### 8. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Methods and Materials

Animals. All animals used in the study were obtained from The Jackson Laboratory, Bar Harbor, Mass., USA. Seventy-two transgenic male B6SJL-Tg(SOD1*G93A)1Gura mice, over-expressing human SOD1 carrying the Gly93→Ala mutation (G93A SOD1) at 7 weeks of age, were randomly assigned to one of four groups receiving different doses of hBM34+ cells or media: Group 1—hBM34+ (5×10⁴ cells/mouse, low dose, n=15), Group 2—hBM34+ (5×10⁵ cells/mouse, mid dose, n=15), Group 3—hBM34+ (1×10⁶ cells/mouse, high dose, n=21), and Group 4—Media (n=21). At 8 weeks of age and then weekly, mice underwent pre-transplant behavioral testing (extension reflex, grip strength test, and rotarod) and monitoring of body weight. When initial disease symptoms appeared such as deterioration of motor function and reduction in body weight (approximately 13 weeks of age), mice intravenously (iv, jugular vein) received either the appropriate hBM34+ cell dose or an equal volume of media. A non-transplant control group (Group 5), consisting of non-carrier mutant SOD1 gene mice from the background strain (control, n=20), only underwent behavioral testing. At 14 weeks of age and weekly thereafter, mice again underwent behavioral testing until 17 weeks of age.

Cell preparation and transplant procedure. Cryopreserved human bone marrow CD34+ cells (hBM34+, AllCells, Alameda, Calif., USA) were thawed rapidly at 37° C. then transferred slowly with a pipette into a centrifuge tube containing 10 ml of Dulbecco's Phosphate Buffered Saline 1× (DPBS), pH 7.4 (Mediatech, Inc., Manassas, Va., USA). The cells were centrifuged (200 g/10 min) at room temperature, the supernatant discarded and the process repeated. After the final wash, cell viability was assessed using the 0.4% trypan blue dye exclusion method before and after transplantation. Transplant cell concentrations were adjusted for each group: 250 cells/μl (5×10⁴ cells/200 μl/injection, Group 1), 2,500 cells/μl (5×10⁵ cells/200 μl/injection, Group 2), and 5,000 cells/μl (1×10⁶ cells/200 μl/injection, Group 3).

The hBM34+ cells were delivered intravenously via the jugular vein of mice under anesthesia with Isofluorane (2-5% at 2L 02/min), as previously described (Garbuzova-Davis et al., J. Hematother. Stem Cell Res. 12, 255-270 (2003); Garbuzova-Davis et al., PLoS ONE 3, e2494 (2008)). Anesthetized animals received a sagittal incision at the base of the neck, and the jugular vein was identified. A 26-gauge needle was inserted into the jugular vein and needle placement verified by a reflux of blood into the syringe. A solution containing the cells was injected during 3 min, and immediately following injection moderate pressure was applied onto the needle vein entry point using a sterile cotton tip. After transplantation, the incision was closed and sutured using monofilament nylon (Ethilon) or a stainless steel wound clip. To assure hemostasis, needle puncture was performed through the muscle overlying the jugular vein, allowing the muscle, combined with digital pressure, to facilitate bleeding stoppage. The media-injected mice in Group 4 received 200 μl of DPBS, the same volume administered to the cell-transplanted mice. Animals in Groups 1-4 received cyclosporine A (CsA, 10 mg/kg ip) daily for the entire post-transplant period.

Tissue Preparation—All animals were sacrificed at 17 weeks of age, 4 weeks after treatment administration. Perfusion was conducted using 0.1 M phosphate buffer (PB) followed by 4% paraformaldehyde (PFA) solution delivered at 80-85 mm Hg of pressure to avoid capillary rupture. The cervical and lumbar segments were then removed and placed in 4% PFA for 24-48 hours. Afterwards, the segments were cryoprotected in 20% sucrose in 0.1 M PB overnight and coronal spinal cord sections were then cut at 30 μm on a cryostat with every 5th section mounted onto a slide to give a series of slides containing sections 150 μm apart. The slides were stored at −20° C. for later staining.

Immunohistochemical staining of hBM34+ cells in the spinal cord. For identification of intravenously transplanted hBM34+ cell engraftment and differentiation potential, serial cervical and lumbar spinal tissue sections from randomly selected mice treated with different cell doses or media (n=5/group) were stained with human anti-Von Willebrand Factor (vWF), an endothelial cell marker, and CD45, a hematopoietic common leukocyte marker. Briefly, the mouse monoclonal antibody (vWF, 1:100, Abcam, USA) was combined with the secondary antibody, monovalent goat anti-mouse Fab′ fragment conjugated to FITC (1:200Jackson ImmunoResearch, USA), and incubated at room temperature (RT) for 2 hours. The tissue sections were pre-incubated with 1% normal human serum (NETS) and 0.5% Triton 100× in PBS for 30 min at RT and subsequently incubated with the previously prepared antibody cocktail overnight at 4° C. Next day, slides were thoroughly washed in PBS and coverslipped with Vectashield containing DAPI (Vector Laboratories, USA). The tissues were then examined under epifluorescence using an Olympus BX60 microscope and images were taken for further analysis of vWF fluorescent immunoexpression. To test for specificity of the immunostaining for vWF and CD45, the primary antibodies were omitted from control slides. No staining was observed in the control sections.

Cervical and lumbar spinal cord tissue sections (n=5/group), were also stained with the human-specific nuclei marker (HuNu). Mouse monoclonal antibody (HuNu, 1:100, Chemicon, USA) was combined with the secondary antibody, monovalent goat anti-mouse Fab′ fragment conjugated to FITC (1:200; Jackson ImmunoResearch, USA), and incubated at room temperature (RT) for 2 hours. Prior to applying this antibody cocktail overnight at 4° C., the tissue sections were incubated in blocking solution for 30 min at RT as described above. The next day, after several rinses in PBS, the tissues were placed in 10% normal goat serum (NGS) and 0.3% Triton 100× in PBS for 60 min at RT and then double-stained with mouse monoclonal antibody for CD45 (1: 200, BD Biosciences Pharmingen, USA) overnight at 4° C. The next day, the slides were rinsed and goat anti-mouse secondary antibody conjugated to rhodamine (1:1000, Alexa, Molecular Probes, USA) was applied for 2 hrs at RT. After several rinses in PBS, the slides were coverslipped with Vectashield containing DAPI (Vector Laboratories, USA). The tissues were then examined under epifluorescence using an Olympus BX60 microscope.

Microhemorrhage staining and analysis Perls' Prussian blue staining was performed on the cervical and lumbar spinal cords from each animal to identify the presence of ferric iron (Fe₃₊) within the parenchyma as an indicator of microhemorrhages. The slides were thawed and hydrated in distilled water for 2 minutes, followed by transfer to 1:1 solution of 10% potassium ferrocyanide (Sigma-Aldrich) and 20% HCl for 20 minutes. After rinsing with distilled water, the slides were counterstained by nuclear-fast red (Sigma-Aldrich) solution for 5 minutes. Slides were then washed in distilled water for 5 minutes and dehydrated in increasing concentrations of ethanol (70%, 80%, 90%, 95%, and 100%) followed by xylene (2×3 minutes), and afterwards coverslipped with Permountrm (Sigma-Aldrich).

Microhemorrhages were observed throughout the cervical and lumbar spinal cord parenchyma using an Olympus BX40 microscope with a SPOT RT3 digital camera (Diagnostic Instruments Inc., Stirling Heights, Mich., USA) under bright field illumination at 20× magnification. Both the left and right sides of every 5th spinal cord section (150 μm apart) were examined. The number and location of microhemorrhages within the cervical and lumbar spinal cord enlargements [38,65] were recorded. The cervical enlargement including C4-C6 segments (14-20 sections/mouse/group) and the lumbar enlargement including L3-L5 segments (10-18 sections/mouse/group) were examined. Within these segmental regions, the gray matter was distinguished from the white matter by the nuclear-fast red counterstain and further defined as the dorsal or ventral horn based on location above or below, respectively, a line perpendicular to the midline passing through the central canal. In the cervical enlargement, the white matter was characterized as anterior (0-0.3 mm from anterior section edge, 0-1.0 mm from midline), posterior (0-0.6 mm from posterior section edge, 0-0.3 mm from midline), or lateral (0-0.4 mm from lateral section edge, 0.2-1.1 mm from posterior section edge). The white matter lumbar enlargement was characterized as follows: anterior (0-0.3 mm from anterior section edge, 0-1.0 mm from midline), posterior (0-0.6 mm from posterior section edge, 0-0.2 mm from midline), and lateral (0-0.3 mm from lateral section edge, 0.3-1.2 mm from posterior section edge). The microhemorrhages were topographically mapped for each mouse accordingly to the mouse spinal cord atlas. Overview of Additionally, white matter microhemorrhages were further defined by the ascending and descending spinal cord pathways.

Characteristics of disease progression. Four measures of animal disease progression were performed blind by independent investigators to avoid subjective bias. Body weight was assessed weekly throughout the study. Extension reflex, rotarod, and grip strength tests started on week 8 and were repeated weekly until 17 weeks of age.

For the extension reflex test the mouse was suspended by the tail and the extension of each hindlimb was observed. If the mouse showed normal hindlimb extension, a score of 2 was given. A score of 1 indicated partial hindlimb extension. If no extension was observed, the score was 0.

In the grip strength test the mouse was held by the tail and carefully placed with all 4 paws on the grid using an instrument to determine grip strength (IDTECH-BIOSEB, France). The animal was gently pulled by the tail and a sensor recorded the force (Newtons, N) with which the mice resisted the pull as a measure of muscle strength. The test was performed three times and the average of the tests was recorded.

For the rotarod test the mouse was placed on a 3.2 cm diameter axle rotating at a speed of 16 rpm (Omnitech Rotoscan, Omnitech Electronics, OH, USA). The latency, in seconds, that the mouse stayed on the rotating axle during a 3 minute maximum period was recorded.

Perfusion and tissue preparation. All cell-treated, media-treated, and control mice were sacrificed at 17 weeks of age (corresponding to 4 weeks after initial treatment at symptomatic disease stage) for immunohistochemical analyses in the cervical and lumbar spinal cords for administered cell differentiation/engraftment and astrocyte expression. Histological analysis of surviving motor neurons was also performed in ventral horns of spinal cords. The mice and controls were injected with 2% Evans Blue dye (EB, Sigma-Aldrich, St. Louis, Mo., USA) in saline solution (4 ml/kg body weight) via the tail vein 30 min prior to perfusion. Based on our previous studies showing that 1×10⁶ stem cell dose is beneficial in treatment of animal models of ALS47 and mucopolysaccharidosis type III B50, spinal cord EB extravasation was evaluated only in mice receiving the high (1×10⁶) cell dose vs. media and control animals. Mice were sacrificed under Euthasol® (0.22 ml/kg body weight) and perfused transcardially with 0.1 M phosphate buffer (PB, pH 7.2) followed by 4% paraformaldehyde (PFA) in PB solution under pressure control fluid delivery at 80-85 mm Hg to avoid capillary rupture. Mice assayed for EB extravasation received only the PB solution. After perfusion, the entire spinal cords were rapidly removed for the EB extravasation assay described below. In remaining mice, the cervical and lumbar spinal cord segments were removed, post-fixed intact in 4% PFA for 24-48 hrs, and then cryoprotected in 20% sucrose in 0.1 M PB overnight. Coronal spinal cord tissues were cut at 30 μm in a cryostat, every fifth section was thaw-mounted onto slides, and the tissue was stored at −20° C. for immunohistochemical and histological analyses.

BSCB permeability. Evans Blue (EB) dye, 961 Da, was used as a tracer for assessing BSCB disruption. After perfusion, mouse spinal cords were weighed and placed in 50% trichloroacetic acid solution (Sigma). Following homogenization and centrifugation, the supernatant was diluted with ethanol (1:3) and loaded into a 96 well-plate in triplicate. The dye was measured with a spectrofluorometer (Gemini EM Microplate Spectrofluorometer, Molecular Devices) at excitation of 620 nm and emission of 680 nm54. Calculations were based on external standards in the same solvent. The EB content in tissue was quantified from a linear standard curve derived from known amounts of the dye and was normalized to tissue weight (m/g). All measurements were performed by two experimenters blinded to the experiment.

Immunohistochemical staining of astrocytes in the spinal cord. Serial sections of the cervical and lumbar spinal cord from randomly selected cell-treated, media-injected, and control mice (n=5/group) were rinsed in PBS to remove the freezing medium. The tissue sections were pre-incubated in a blocking solution of 10% NGS and 3% Triton 100× in PBS for 60 min at RT, followed by overnight incubation with rabbit polyclonal anti-glial fibrillary acidic protein primary antibody (GFAP, 1:500, Dako, Denmark) at 4° C. On the next day, slides were rinsed in PBS and incubated with goat anti-rabbit secondary antibody conjugated to FITC (1:500, Alexa, Molecular Probes, USA) for 2 hrs at RT. After several rinses in PBS, the slides were coverslipped with Vectashield containing DAPI (Vector Laboratories, USA). The tissues were then examined under epifluorescence using an Olympus BX60 microscope and images were taken for further analysis of GFAP fluorescent immunoexpression.

Immunohistochemical staining of microglia in the spinal cord. Tissue sections were rinsed in PBS to remove the freezing medium and pre-incubated in a blocking solution of 4% NGS and 2% Triton 100× in PBS for 60 min at RT, followed by overnight incubation with rabbit polyclonal anti-ionized calcium binding adapter molecule-1 primary antibody (Iba-1, 1:500, Wako Chemicals, Richmond, Va., USA) at 4° C. On the next day, slides were rinsed in PBS and incubated with goat anti-rabbit secondary antibody conjugated to rhodamine (1:500, Alexa, Molecular Probes, USA) for 2 hrs at RT. After several rinses in PBS, the slides were coverslipped with Vectashield containing DAPI (Vector Laboratories, USA). The tissues were then examined under epifluorescence using an Olympus BX60 microscope and images were taken for further analysis of Iba-1 fluorescent immunoexpression.

vWF and astrocytic immunoexpression analyses in the spinal cord. Analyses of vWF and GFAP fluorescence immunoexpressions in the cervical and lumbar spinal cords from 17-week-old mice were performed in the ventral horns by an investigator blinded to the experiments. Animal codes were removed prior to analysis. Immunohistochemical image analyses for vWF and GFAP were performed by measuring intensity of fluorescent expression (%/mm2) in NIH ImageJ (version 1.46) software. Thresholds for detection of vWF and GFAP fluorescent expressions were adjusted for each image to eliminate background noise. To avoid bias in the analysis of fluorescent images, specific spinal cord areas were identified in a section using a 106/0.30 numerical aperture (NA) lens, and then areas of interest were photographed with either a 206/0.50 NA or 406/0.75 NA lens, photographing the slide in a random raster pattern. Measurements of cervical/lumbar ventral horn area were first performed by determining the cross-point of a line passing the central canal perpendicular to the midline. The area of ventral gray matter was determined below this line in the right and left cervical/lumbar spinal cords in coronal sections from each mouse group at predetermined uniform intervals (150 mm). For detection of vWF immunopositive cells in cell-treated mice, immunohistochemical images taken in randomly selected areas from right and left ventral gray matter of the cervical and lumbar spinal cords at 40×. Fluorescent intensity (%/mm²) was measured in the entire image. Data are presented as averages of vWF cell immunoexpression of both sides. For GFAP intraparenchymal immunoexpressions in cell-treated, media-injected, and control mice, immunohistochemical images were taken from right and left ventral gray matter of the cervical and lumbar spinal cords at 10×. Density of astrocytes was determined as percentage per mm² separately for the cervical and lumbar spinal cord sites. Also, fluorescent images of GFAP immunoexpression were taken of lateral and anterior white matter from both sides of the cervical/lumbar spinal cords at 10× for analyses. GFAP perivascular immunoexpression was analyzed in the cervical/lumbar ventral horns of both sides from cell-treated, media-injected, and control mice. Fluorescent GFAP intensity of astrocytic end-feet (perivascular astrocytes) was measured adjacent to abluminal side of capillaries of approximately 25-30 μm in diameter. Density of perivascular GFAP immunoexpressions was determined as percentage per mm² separately for cervical and lumbar spinal cord sites.

Histological staining and stereological count of motor neurons in the spinal cord. A separate set of cervical and lumbar spinal cord sections from randomly selected mice from each group were stained with 0.1% cresyl violet using a standard protocol for examination of motor neuron condition for the Nissl substance. Motor neuron numbers in the ventral horn of the cervical and lumbar spinal cords were determined by the optical fractionator method of unbiased stereological cell counting techniques using a Nikon Eclipse 600 microscope and quantified by using Stereo Investigator® software (MicroBrightField). The virtual grid (150×150 μm) and counting frame (75×75 μm) were optimized to count at least 200 cells per animal with error coefficients <0.07. Outlines of the anatomical structures were done using 10×/0.45 objective, and cell quantification was conducted using 40×/1.40 objective. The motor neuron numbers (20-25 μm diameter) were counted in discrete levels of the cervical (C1-C3, C4-C6, and C7-C8) and lumbar spinal (L1-L2, L3-L4, and L5-L6) cords (n=7 sections/level/spinal cord segment/group separated by approximately 120 μm) and presented as averages per ventral horn for both spinal cord sides. Motor neuron morphologies were also analyzed in the cervical and lumbar spinal cords.

Statistical analysis. Data are presented as means±S.E.M. One-way ANOVA with Tukey's Multiple Comparison test using GraphPad Prism software version 5 (GraphPad Software) was performed for statistical analysis.

### Example 2

### Microhemorrhages within the Spinal Cords of G93A Mice

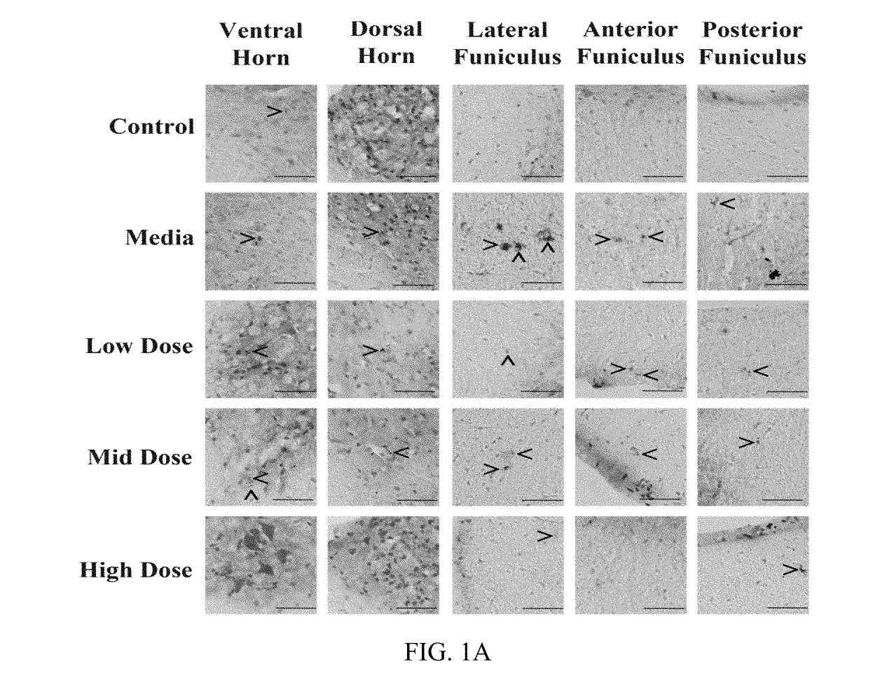

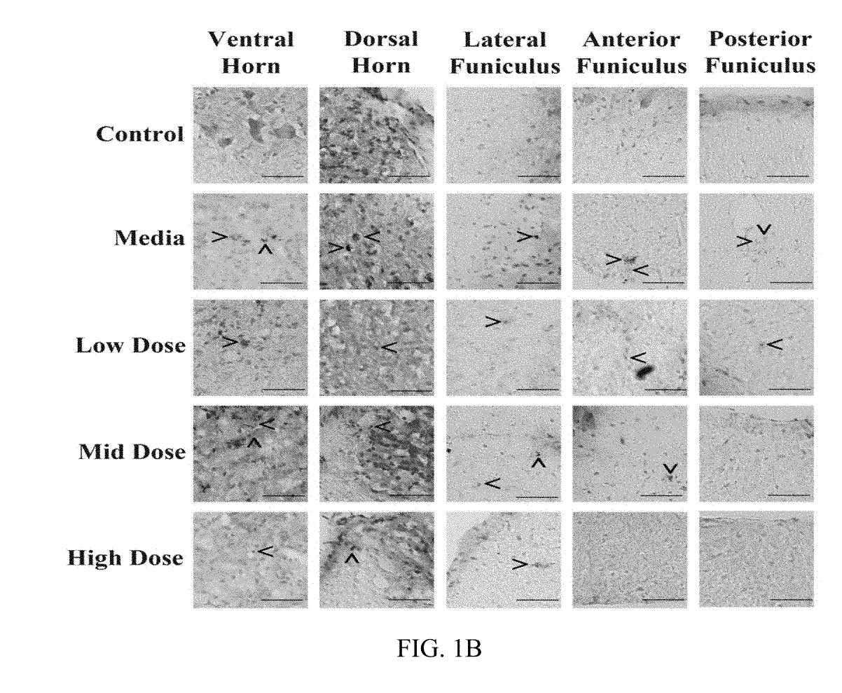

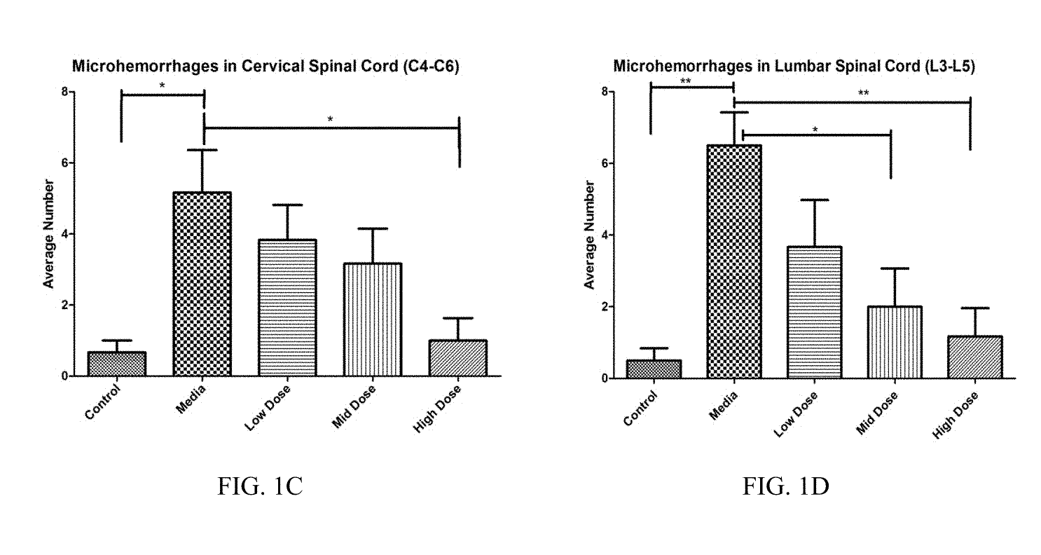

Perl's Prussian blue staining revealed ferric iron deposits, consequences of microhemorrhages, within the gray and white matter parenchyma of the cervical spinal cord in all examined animals to different degrees. While microhemorrhages were rare in the control mice (FIG. 1A), numerous microhemorrhages were observed in media-treated mice in all five evaluated regions (FIG. 1A). The microhemorrhages varied in size within each animal group with more detected in the lateral and anterior white matter of media-treated animals. Similarly to media-treated mice, ferric iron deposits were observed in analyzed spinal cord regions of the low (FIG. 1A) and mid (FIG. 1A) cell dose-treated mice. However, no microhemorrhages were detected within the ventral horn (FIG. 1A), dorsal horn (FIG. 1A), or anterior white matter (FIG. 1A) of the high cell dose-treated mice.

Quantitative analysis of microhemorrhages in the cervical spinal cords determined a significantly (p<0.05) higher number of microhemorrhages in the media-treated mice compared to controls (FIG. 1C). Cell-dose-dependent decreases in the number of microhemorrhages were noted after hBM34+ cell transplantation compared to the media-treated mice, reductions which reached significance (p<0.05) in the high cell-dose mice. In the media-treated mice, the percentages of microhemorrhages decreased across the regions as follows: ventral horn (35.5%), dorsal horn (22.6%), anterior white matter (22.6%), lateral white matter (16.1%) and posterior white matter (3.2%) (Table 1). A similar pattern of microhemorrhage distribution was also seen in the low and mid dose-treated mice, however, only a few iron deposits were detected in ALS mice treated with high cell-dose. Importantly, no microhemorrhages were detected in 50% of the high cell-dose treated mice, although microhemorrhages were observed in 100% of the media, low, and mid cell-dose mice. The number of microhemorrhages within the gray matter was greater vs. the white matter in media-treated, low, and mid cell-dose treated mice, with the exception of the high cell-dose mice.

Similarly to the cervical spinal cords, microhemorrhages were rarely detected in the control mice (FIG. 1B), but numerous microhemorrhages were observed throughout the lumbar spinal cord of the media-treated mice in all analyzed regions (FIG. 1B). The microhemorrhages varied in size within each animal group. In media-treated mice, numerous large microhemorrhages were determined mainly in the ventral horn (FIG. 1B), dorsal horn (FIG. 1B), and anterior white matter (FIG. 1B). Ferric iron deposits were observed in all analyzed spinal cord regions of the low cell-dose treated mice (FIG. 1B). In mid cell-dose treated mice, microhemorrhages were detected in the gray matter (FIG. 1B) and lateral or anterior white matter (FIG. 1B) but not in the posterior white matter region (FIG. 1B). However, microhemorrhages were identified in the gray matter (FIG. 1B) and lateral white matter (FIG. 1B) of high cell-dose treated mice. In the anterior (FIG. 1B) and posterior (FIG. 1B) white matter of these treated mice no iron deposits were observed.

Significantly (p<0.01) more microhemorrhages were observed in the lumbar spinal cord of the media-treated mice compared to the controls (FIG. 1D). Cell-dose dependent decreases in microhemorrhage numbers in comparison to media-treated mice were determined after hBM34+ cell transplantation, reaching significance in both the mid (p<0.05) and high cell-dose (p<0.01) mice (Table 1).

In the media-treated mice, the percentage of microhemorrhages within each region of the lumbar spinal cord decreased as follows: ventral horn (41.0%), dorsal horn (28.2%), lateral white matter (15.4%), anterior white matter (10.3%), and posterior white matter (5.1%) (Table 1). A similar pattern of microhemorrhage distribution was also seen in the low dose mice. No microhemorrhages were observed in the posterior white matter of the mid dose mice or the anterior and posterior white matter of the high cell-dose mice. Overall, no microhemorrhages were detected in 33% and 50% of the mid and high dose mice respectively, while microhemorrhages were observed in 100% of the media and low dose mice. The number of microhemorrhages within the gray matter was higher vs. white matter in media-treated and all cell treated mice.

Topographic distribution of microhemorrhages in the cervical and lumbar spinal cords of media-treated and cell-treated mice was analyzed by visual detection of the gray and white matter tissues accordingly to the mouse spinal cord atlas and the ascending/descending pathways as described herein. Microhemorrhage locations in the C4-C6 and L3-L5 spinal cord enlargements were mapped onto each analyzed spinal cord segment. FIG. 1E presents overall microhemorrhage distribution in the gray and white matter for each animal group at C4 and L4 segments.

In the cervical spinal cords of control mice, a few microhemorrhages were detected in the gray and white matter (FIG. 1E). Within the ventral horn of media-treated mice, the majority of microhemorrhages were noted in the ventral horn of lamina 7Sp and some were observed in lamina 9Sp (FIG. 1E). The number of microhemorrhages decreased within these regions of the cervical spinal cord in a cell-dose dependent fashion after hBM34+ cell transplantation. However, numerous iron deposits were still identified in low and mid cell-dose treated mice, mainly at level of lamina 7Sp (FIG. 1E). No microhemorrhages were determined at level of lamina 9Sp and only a few iron deposits were observed at lamina 7Sp in mice receiving the high cell-dose (FIG. 1E).

Microhemorrhages within the cervical dorsal horn of media-treated mice were primarily localized in the 3Sp, 4Sp, and 5Sp laminas (FIG. 1E). Microhemorrhage numbers decreased with escalating cell doses (FIG. 1E). In high cell-dose treated mice no microhemorrhages were observed within the dorsal horn (FIG. 1E). In the cervical white matter of the media-treated mice, many microhemorrhages were determined in the lateral and anterior white matter (FIG. 1E) corresponding to areas of ascending and descending pathways, such as the spinothalamic and reticulospinal tracts. Fewer iron deposits were found in low and mid cell-dose treated mice (FIG. 1E). No microhemorrhages were observed in the high cell-dose mice within the anterior white matter area related to the descending pathways (FIG. 1E).

In contrast to the cervical spinal cord, the topographic distribution of the microhemorrhages within the ventral horn of the lumbar spinal cord in media-treated mice revealed similar locations in laminas 7Sp and 9Sp (FIG. 1E). A dose-dependent decrease in microhemorrhages was evident in mice treated with low and mid cell-doses (FIG. 1E). Only a few microhemorrhages were present at lamina 9Sp in the high-dose mice (FIG. 1E). In the dorsal horn of the lumbar spinal cord, microhemorrhages were found throughout laminas 1Sp-6Sp in the media-treated mice (FIG. 1E). The overall cell-dose dependent decrease of microhemorrhages in the lumbar spinal cord mirrored patterning of iron deposits in the cervical dorsal horn (FIG. 1E). Only a small number of microhemorrhages were found within laminas 2Sp and 3Sp of the high cell-dose treated mice (FIG. 1E). Within the lumbar white matter of the media-treated mice, noted microhemorrhages corresponded with the locations of ascending (spinocerebellar and spinothalamic) and descending (corticospinal and reticulospinal) pathways (FIG. 1E). In the lumbar white matter of the ALS mice treated with low or mid cell doses, a few iron deposits were determined, mainly within the lateral white matter corresponding to ascending pathways (FIG. 1E). In high cell-dose treated mice, microhemorrhages were only observed in the area of the lateral spiny nucleus (FIG. 1E).

TABLE 1
Distribution of microhemorrhages in the cervical and lumbar spinal cords.

| | | Number/Percentage of Microhemorrhages | | | | |
|---|---|---|---|---|---|---|
| | | by Spinal Cord Region | | | | |
| | Average Number of | | | Lateral | Anterior | Posterior |
| Animal | microhemorrhages ± | Ventral | Dorsal | White | White | White |
| Group | S.E.M. | Horn | Horn | Matter | Matter | Matter |

Cervical Spinal Cord

| | | | | | | |
|---|---|---|---|---|---|---|
| Control | 0.67 ± 0.33 | 2/50.0 | 0/0.0 | 1/25.0 | 1/25.0 | 0/0.0 |
| Media | 5.17 ± 1.19 | 11/35.5 | 7/22.6 | 5/16.1 | 7/22.6 | 1/3.2 |
| Low Dose | 3.83 ± 0.98 | 7/30.5 | 5/21.7 | 5/21.7 | 5/21.7 | 1/4.4 |
| Mid Dose | 3.17 ± 0.98 | 6/31.6 | 5/26.3 | 2/10.5 | 4/21.1 | 2/10.5 |
| High | 1.00 ± 0.63 | 2/33.3 | 0/0.0 | 1/16.7 | 0/0.0 | 3/50.0 |
| Dose | | | | | | |

Lumbar Spinal Cord

| | | | | | | |
|---|---|---|---|---|---|---|
| Control | 0.50 ± 0.34 | 1/33.4 | 1/33.3 | 0/0.0 | 1/33.3 | 0/0.0 |
| Media | 6.50 ± 0.92 | 16/41.0 | 11/28.2 | 6/15.4 | 4/10.3 | 2/5.1 |
| Low Dose | 3.67 ± 1.31 | 10/45.5 | 6/27.3 | 3/13.6 | 2/9.1 | 1/4.5 |
| Mid Dose | 2.00 ± 1.06 | 4/33.3 | 3/25.0 | 3/25.0 | 2/16.7 | 0/0.0 |
| High | 1.17 ± 0.79 | 3/42.8 | 2/28.6 | 2/28.6 | 0/0.0 | 0/0.0 |
| Dose | | | | | | |

### Example 3

### Immunohistochemical Analysis of Administered hBM34+ Cells In Vivo

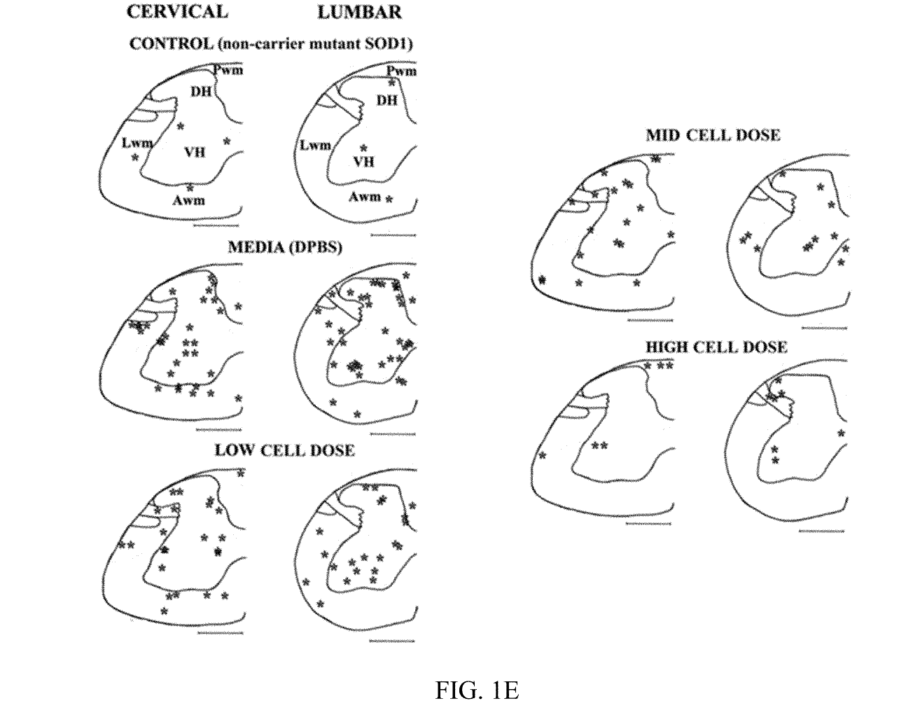

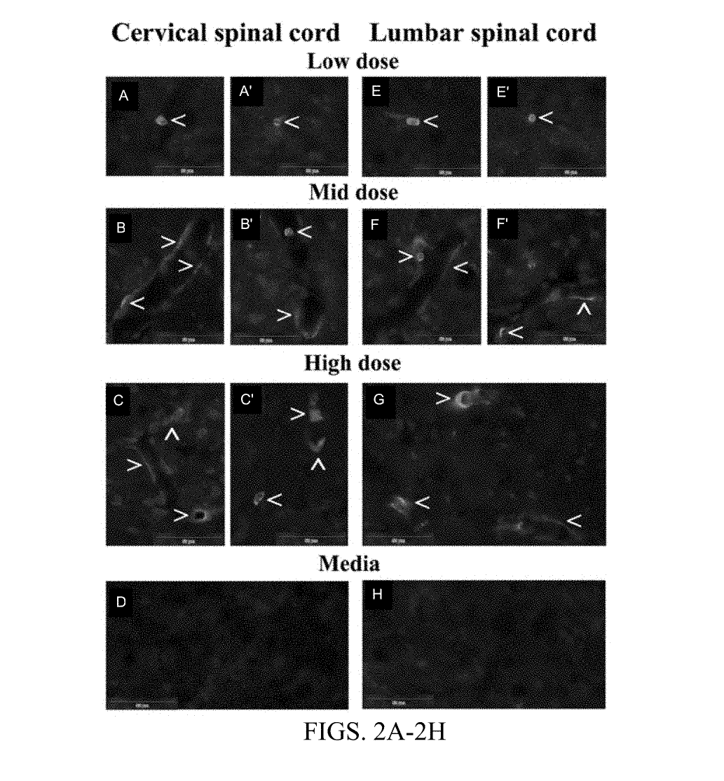

Four weeks after intravenous hBM34+ cell transplantation, the cervical and lumbar tissues from mice treated with different cell doses were immunohistochemically stained with human anti-Von Willebrand Factor (vWF), an endothelial cell marker. Cellular vWF immunoexpression was identified mainly in the ventral spinal cord horns of all cell-treated ALS mice. In mice receiving the low cell dose, most vWF positive cells were rounded or oval shaped and congregated in capillaries in both cervical (FIGS. 2A,2A′) and lumbar (FIGS. 2E,2E′) spinal cords. Adherence of transplanted cells to the lumen of microvessels by forming a distinguishable line in the capillary walls was determined in ALS mice with mid cell dose by vWF immunoexpression in the cervical (FIGS. 2B,2B′) and lumbar (FIGS. 2F,2F′) spinal cords. Numerous capillaries in the cervical (FIGS. 2C,2C′) and lumbar (FIG. 2G) spinal cords of mice receiving the high cell dose showed immunopositive vWF expression in vascular walls, indicating superior adherence of transplanted cells. Positive immunoexpression of vWF was also indicated in dorsal horn and white matter microvessels. Of note, cellular vWF immunoexpression was not found in the parenchyma of spinal cords from cell-treated mice or in capillaries/parenchyma from media-treated ALS mice (FIGS. 2D,2H). Quantitative analysis of fluorescent vWF expression in randomly selected areas from right and left ventral gray matter of the cervical and lumbar spinal cords demonstrated significantly increased intensities of vWF fluorescent expression corresponding with higher transplant cell doses (FIG. 2I).

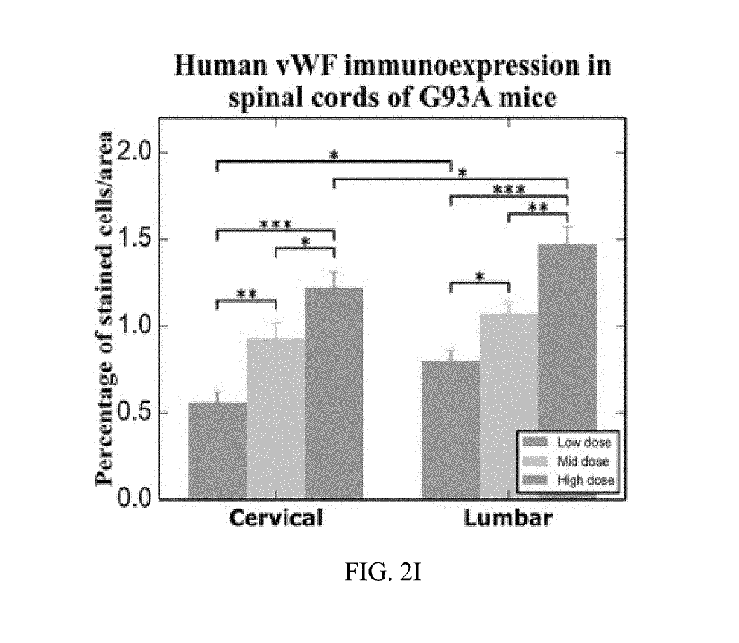

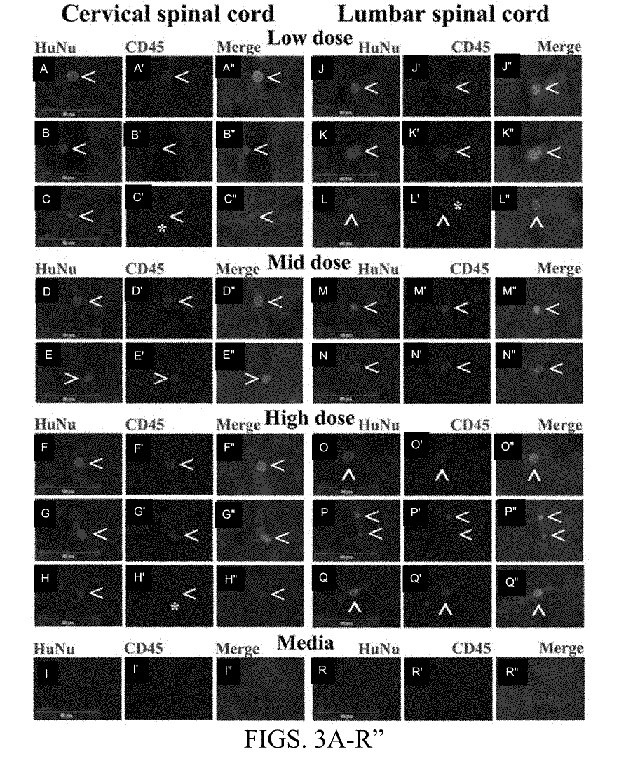

In a separate set of the cervical and lumbar tissues from cell-treated mice, double immunohistochemical staining was performed with the human-specific nuclei (HuNu) and CD45, a hematopoietic common leukocyte marker. In the cervical spinal cord, double immunopositive HuNu+/CD45+ cells were determined in mice treated with low (FIGS. 3A-3B″), mid (FIGS. 3D-3E″), and high (FIGS. 3F-3G″) cell doses. These cells are predominantly of rounded morphology and located within the capillary lumen. A few cells were HuNu+/CD45-(FIGS. 3C-3C″,3H-3H″) and were found some distance from blood vessels. Similarly to the cervical spinal cord, immunopositive HuNu+/CD45+ cells were found in the lumbar spinal cords of all cell-treated mice: low (FIGS. 3J-3K″), mid (FIGS. 3M-3N″), and high (FIGS. 3O-3Q″) cell doses. Also, some cells only immunoexpressed HuNu and were negative for CD45 (FIGS. 3L-3L″). No cells expressing HuNu or CD45 were determined in cervical (FIGS. 3I-3I″) or lumbar (FIGS. 3R-3R″) spinal cords from media-injected mice.

Thus, immunohistochemical analysis in vivo showed that transplanted cells differentiated into endothelial cells and had engrafted into capillaries in the cervical and lumbar spinal cords by 4 weeks post-transplant. However, some administered cells expressed CD45 common leukocyte antigen and some cells were positive only for HuNu, suggesting differentiation of transplanted cells into cells with different immunophenotypes.

### Example 4

### Effect of hBM Transplantation on the Behavioral Outcomes of a Mouse Model of ALS

Of the 64 total G93A ALS symptomatic mice used in the study, five mice were excluded due to death precipitated by conditions other than disease progression, more specifically, anesthetic complications during cell or media administrations. Also, four mice were found dead at 16 or 17 weeks of age. Data on behavioral outcomes of these animals were included in analyses.

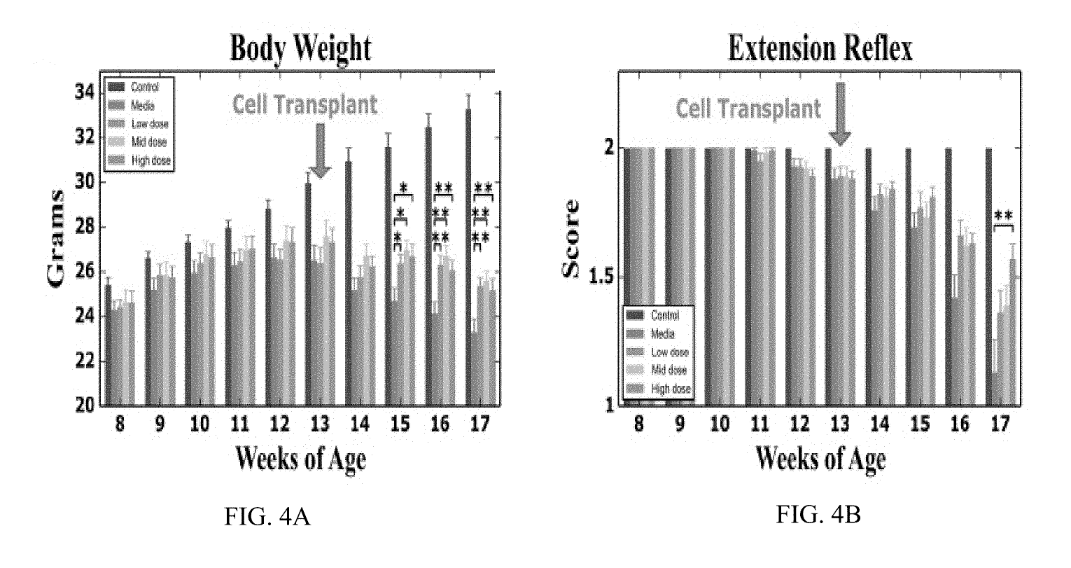

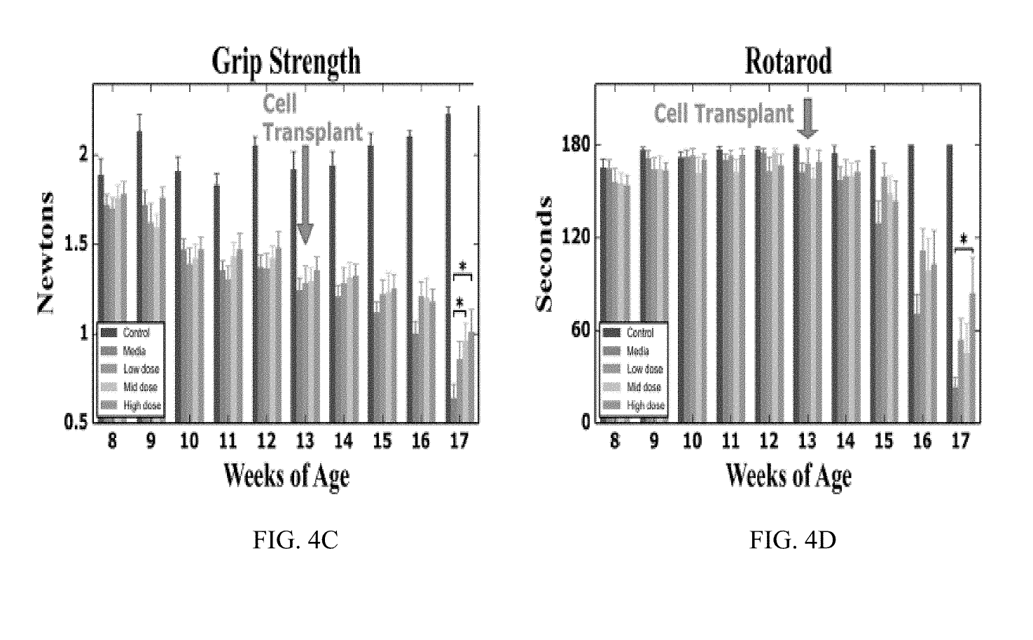

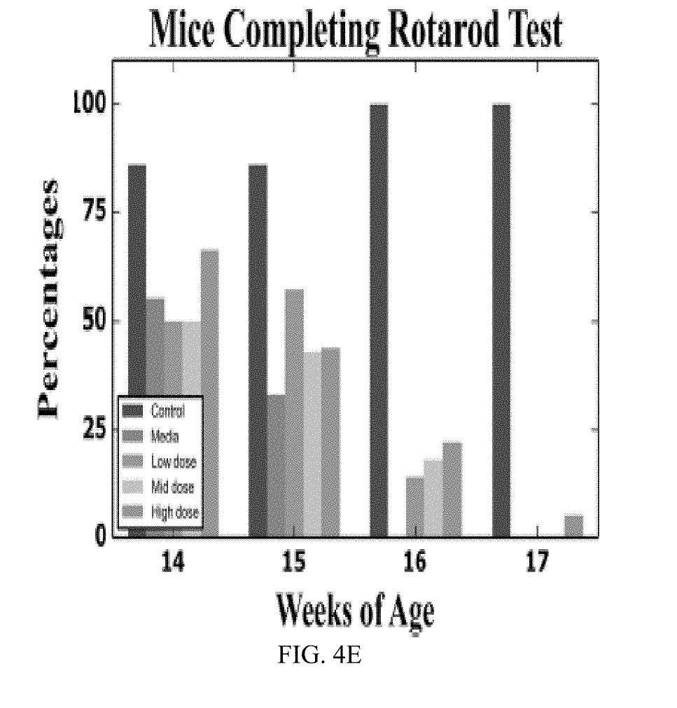

Body weight, a general indicator of mouse health, and also a valuable marker for detecting progression of muscle atrophy, was measured weekly. As expected, body weight started to slowly decline at the symptomatic age of approximately 13-14 weeks in media-treated G93A mice and by 17 weeks of age, these mice had lost about 12% of their maximum body weight for this period. Although hBM34+ cell-treated animals lost weight more slowly, there were no significant differences between media and cell-treated mice at 1 week post-transplant. Significantly (p<0.05) higher body weights were determined in all cell-treated mice at 2 weeks post-transplant (15 weeks of age) compared to media-injected mice (FIG. 4A). At 3 and 4 weeks after cell transplantation (16 and 17 weeks of age), all cell-treated mice maintained significantly higher body weights (p<0.01) than media-injected mice. Of note, there were no significant differences in body weights between mice treated with different cell doses during the entire post-transplant period and at 17 weeks of age cell-treated mice weighed 2-2.5 grams more than media-injected mice.

Cell-treated mice also displayed superior performance in tests of functional ability. Deteriorating extension reflex was noted in media-treated G93A mice, beginning at 12-13 weeks of age (1.93±0.03 and 1.88±0.04 score, respectively), with extension progressively declining until 17 weeks of age (1.13±0.13 score). However, hindlimb extension of mice treated with cells deteriorated more slowly than media-injected mice. At 16 weeks of age (3 weeks post-transplant), mice receiving cell transplantation displayed a tendency towards delayed deterioration of hindlimb extension compared to media-injected mice (FIG. 4B). At 17 weeks of age, only mice receiving 1×10⁶ cells showed significantly (p<0.05) higher extension reflex scores vs. media-injected mice. Also, these mice demonstrated hindlimb extension scores (1.63±0.04) superior to mice receiving 5×10⁴ cells (1.36±0.09) or 5×10⁵ cells (1.39±0.08).

In the grip strength test, media-treated G93A mice started to show decreased muscle strength at approximately 13 weeks of age (1.24±0.07 N), with strength progressively declining during the course of disease until 17 weeks of age (0.64±0.08 N) (FIG. 4C). Delayed loss in muscle strength was determined mainly in all cell-treated mice vs. media-injected at 15-16 weeks of age with significance (p<0.05) in mice with 5×10⁵ cells (0.96±0.10 N) and 1×10⁶ cells (1.01±0.13 N) at 17 weeks of age (FIG. 4C). Although muscle strength in mice receiving 5×10⁴ cells (0.86±0.10 N) was superior to media-injected mice at 17 weeks of age, no significant difference between these groups was determined.

Declines in performance on the rotarod test were observed in media-treated mice starting at week 13. Cell-treated mice demonstrated longer latencies for the entire post-transplant period vs. media-injected mice. At 17 weeks of age, ALS mice receiving 1×10⁶ cells showed significantly (p<0.05) higher rotarod latency (84.07±23.22 sec) compared to media-injected (23.28±6.25 sec) or other cell-treated mice (5×10⁴ cells: 53.85±13.66 sec; 5×10⁵ cells: 45.23±19.12 sec) (FIG. 4D). Importantly, at 16 weeks of age, 14, 19, or 22%, of mice transplanted with low, mid, or high cell dose respectively were able to complete the rotarod test (180 sec) while media-injected mice could not complete the test (FIG. 4E). At 17 weeks of age, about 6% of mice receiving the high 1×10⁶ cell dose were the only animals to complete the test.

Thus, intravenous transplantation of hBM34+ cells at different doses into symptomatic ALS mice ameliorated behavioral outcomes during 4 weeks post-treatment vs. media-injected mice by maintaining body weight and delaying losses in hindlimb extension, muscle strength, and rotarod performance. However, the most beneficial effect on motor function was determined in G93A mice treated with high 1×10⁶ cell dose.

### Example 5

### Effect of hBM34+ Cell Transplantation at Different Doses on Protoplasmic Astrocytes

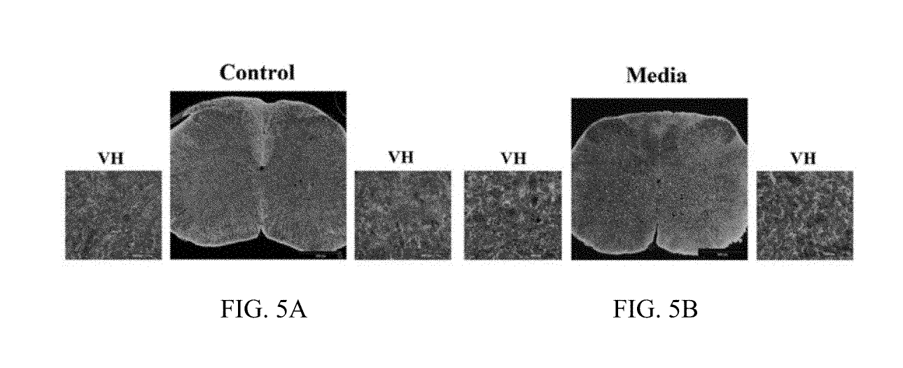

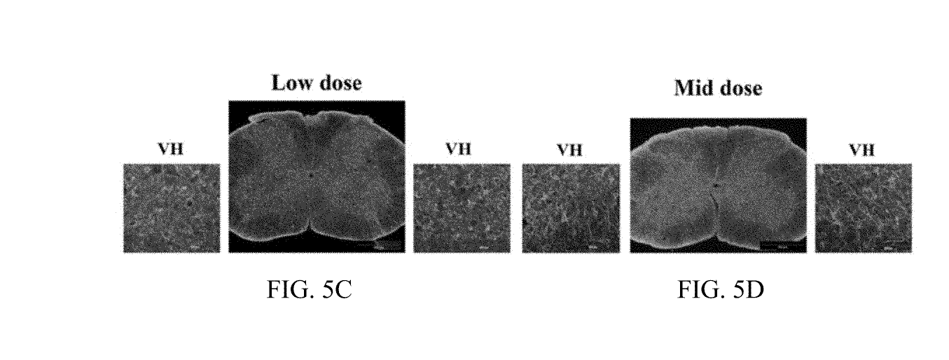

Immunohistochemical analysis of astrocytes in the cervical and lumbar spinal cords from cell-treated, media-injected, and control mice at 17 weeks of age was performed by fluorescent immunostaining with GFAP, an intermediate filament protein. In the cervical spinal cord, protoplasmic astrocytes with large cell bodies and hypertrophic processes at high density were determined in media-injected mice (FIG. 5B), while control mice presented astrocytes with fine cellular processes, distributed relatively uniformly within the spinal cord gray matter (FIG. 5A). In ALS mice treated with low cell dose, a moderate decrease of reactive astrocytes was noted in the ventral horns (FIG. 5C). Fewer reactive astrocytes and more astrocytes with thin cell processes were seen in the gray matter of the cervical spinal cords of ALS mice with mid dose (FIG. 5D). Only a few astrocytes with thick cell processes and enormous cell bodies were found in mice after high cell dose transplantation (FIG. 5E). In these mice, more astrocytes with normal morphology were noted. Quantitative analysis of GFAP immunoexpression reflects astrocytic morphology in the cervical ventral horns of analyzed animals and reveals significantly (p<0.001) higher fluorescent expression of GFAP in media-injected mice (36.21±0.92%) vs. controls (7.83±0.26%) (FIG. 5F). A significant (p<0.001) decrease of GFAP immunoexpression was determined in all cell-treated compared to media-injected mice. Fluorescent GFAP expressions were inversely proportional to cell doses: low: 31.26±0.71%, mid: 26.05±0.46%, and high: 21.85±0.46% cell doses (FIG. 5F).

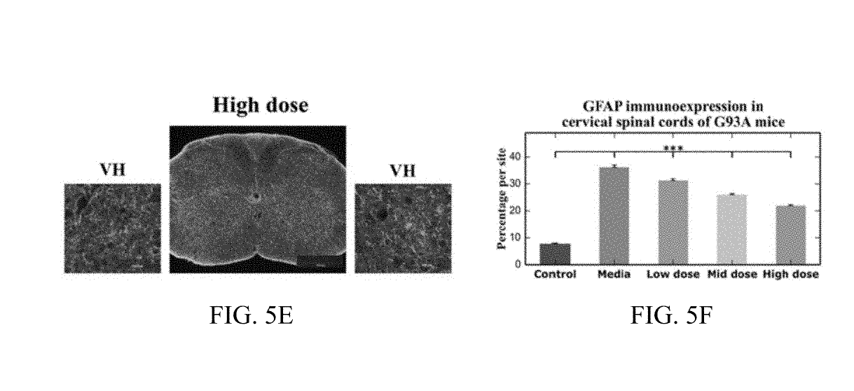

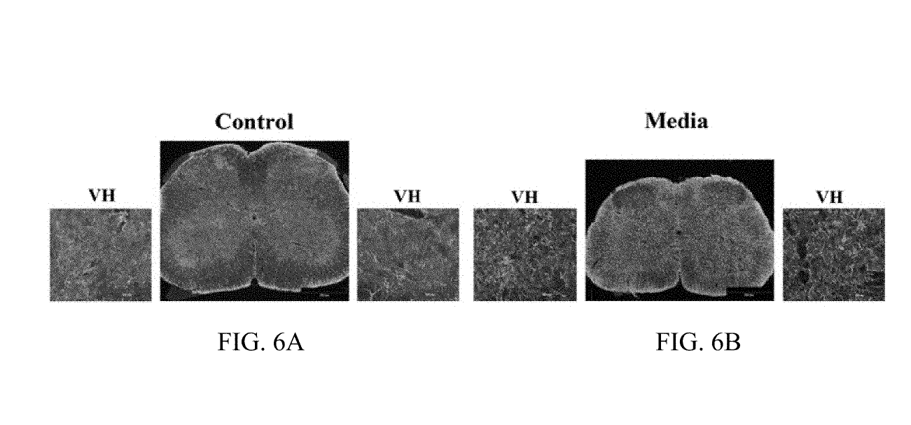

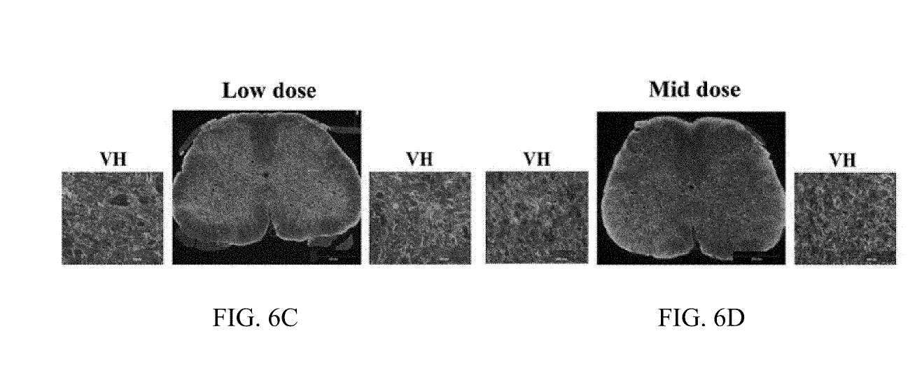

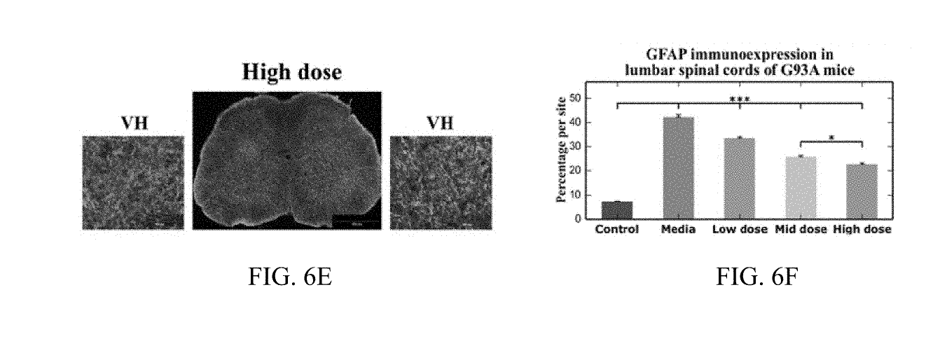

Similarly to the cervical spinal cord, astrocytic morphological profiles in the lumbar spinal cords from cell-treated, media-injected, and control mice at 17 weeks of age were observed. Media-treated ALS mice showed astrocytosis as indicated by protoplasmic astrocytes with large cell bodies and thick processes (FIG. 6B) compared to controls with typical astrocyte morphology (FIG. 6A). In ALS mice treated with low (FIG. 6C), mid (FIG. 6D), or high (FIG. 6E) cell doses, astrocyte cell reactivity was reduced in the ventral horns. Many astrocytes with distinctive fine cell processes at low density were detected in these animals. The measurements of GFAP immunoexpression in the lumbar ventral horns demonstrated a significant (p<0.001) increase of GFAP fluorescent expression in media-injected mice (42.20±1.09%) versus controls (7.19±0.27%) (FIG. 6F). A significantly (p<0.001) decreased rate of GFAP immunoexpression was associated with elevated cell doses: low: 33.39±0.66%, mid:25.90±0.66%, and high: 22.68±0.63% cell doses (FIG. 6F). Of note, the difference between high and mid doses was significant at p<0.05.

Thus, immunohistochemical analysis of GFAP immunoexpression in the cervical and lumbar spinal cords showed substantial astrogliosis in media-injected mice at 17 weeks of age. In these mice, reactive protoplasmic astrocytes were characterized by large cell bodies and hypertrophic processes. Four weeks after cell transplantation, astrocyte cell reactivity was significantly reduced in cell-treated mice and decreased rates of astrocytosis were associated with elevated cell doses.

### Example 6

### Effect of hBM34+ Cell Transplantation at Different Doses on Fibrous Astrocytes

In the same set of cervical and lumbar spinal cord sections from 17-week-old cell-treated, media-injected, and control mice used for protoplasmic astrocyte analysis, fluorescent images of GFAP immunoexpression of fibrous astrocytes were obtained from lateral and anterior white matter of both sides of the spinal cord at 10× magnification. In the cervical spinal cord, fibrous astrocytes were typically oriented in lateral and anterior white matter in control mice. Reactive fibrous astrocytes with hypertrophic processes were mainly determined in the lateral column of media-injected mice. In some areas of anterior white matter, thick astrocyte processes were observed. Fibrous astrocyte reactivity in both lateral and anterior white matter was reduced in cell-treated mice proportionally with elevated cell doses. However, some reactive astrocytes were determined in lateral spinal column in mice treated with low or mid cell doses. Similarly to the cervical spinal cord, fibrous astrocytes in analyzed white matter lumbar spinal cord regions of control mice showed normal morphology. A substantial increase of GFAP immunoexpression was found in the lateral columns of media-injected mice. Although a lesser degree of GFAP immunoexpression was seen in this white matter area of cell-treated mice, regional density of hypertrophic astrocyte processes was observed. In anterior white matter of the lumbar spinal cord, few thick fibrous astrocyte processes were determined in media-injected and low cell-treated mice.

Thus, GFAP immunoexpression in lateral and anterior white matter in the cervical and lumbar spinal cords showed a substantial increase of reactive fibrous astrocytes in media-injected mice at 17 weeks of age. Four weeks after cell transplantation, reduced astrocyte cell reactivity in areas of white matter tracts in cell-treated mice was associated with increased cell doses.

### Example 7

### Effect of hBM34+ Cell Transplantation at Different Doses on Perivascular Astrocytes

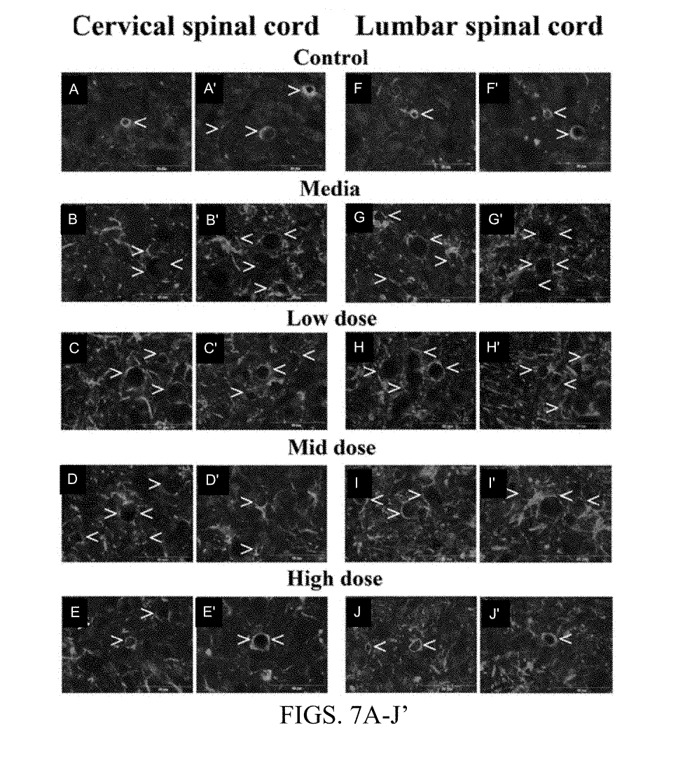

In the same set of cervical and lumbar spinal cord sections from 17-week-old cell-treated, media-injected, and control mice used for protoplasmic astrocyte analysis, fluorescent images of GFAP immunoexpression of perivascular astrocytes were obtained from ventral horns of both sides of the spinal cord. Normal appearance of delineated perivascular astrocytes fully covering capillaries was observed in the cervical (FIGS. 7A,7A′) and lumbar (FIGS. 7F,7F′) spinal cords from control animals. Perivascular astrocytes surrounding capillaries were partially revealed in media-injected mice in both the cervical (FIGS. 7B,7B′) and lumbar (FIGS. 7G,7G′) spinal cords. Also, reactive protoplasmic astrocytes near capillaries were noted in these animals. In mice receiving low or mid cell doses, there were no substantial changes in presence of perivascular astrocytes from cervical (low dose; FIGS. 7C,7C′; mid dose:FIGS. 7D,7D′) or lumbar (low dose; FIGS. 7H,7H′; mid dose: FIGS. 7I,7I′) spinal cords compared to media-injected mice. Only a few capillaries with regularly delineated perivascular astrocytes were observed in spinal cord capillaries from mice receiving the mid cell dose. In contrast, numerous capillaries with typical perivascular astrocytes surrounding capillaries were determined in both cervical (FIGS. 7E,7E′) and lumbar (FIGS. 7J,7J′) spinal cords of high-cell-dose mice. Analysis of fluorescent GFAP perivascular immunoexpression showed similar significant (p<0.001) decreases of GFAP intensity in both cervical (18.30±1.14%) and lumbar (19.75±1.06%) spinal ventral horns from media-injected mice compared to control mice: cervical: 71.95±2.12% and lumbar: 73.39±1.99% (FIG. 7B). In the cervical spinal cord, significant increases of perivascular GFAP immunoexpression were determined in mice receiving mid (p<0.01, 25.60±1.26%) and high (p<0.001, 37.28±1.77%) cell doses vs. media-injected animals. Also, GFAP intensity in high-cell-dose mice was significantly (p<0.001) greater compared to low or mid cell doses. In the lumbar spinal cord, no significant differences in perivascular GFAP fluorescent expression were found between media-injected (19.75±1.06%), low (20.34±1.04%), and mid (23.60±1.11%) cell doses. Only mice transplanted with high cell dose demonstrated a significant (p<0.001) increase of perivascular immunoexpression (35.64±1.49%) in comparison to mice from media-injected, low, or mid cell doses groups (FIG. 7K).

Thus, immunohistochemical analysis of perivascular GFAP immunoexpression showed significant decreases in delineated astrocytes and their perivascular end-feet at the blood capillaries in the cervical and lumbar spinal ventral horns in media-injected mice at 17 weeks of age. Four weeks after cell transplantation, perivascular astrocytes were re-established in numerous capillaries, mainly in mice treated with high cell dose in both the cervical and lumbar spinal cords. Also, mice receiving mid cell dose showed significantly increased perivascular GFAP immunoexpression only in the cervical spinal cords vs. media-injected animals.

### Example 8

### Effect of hBM34+ Cell Transplantation at Different Doses on Microglia

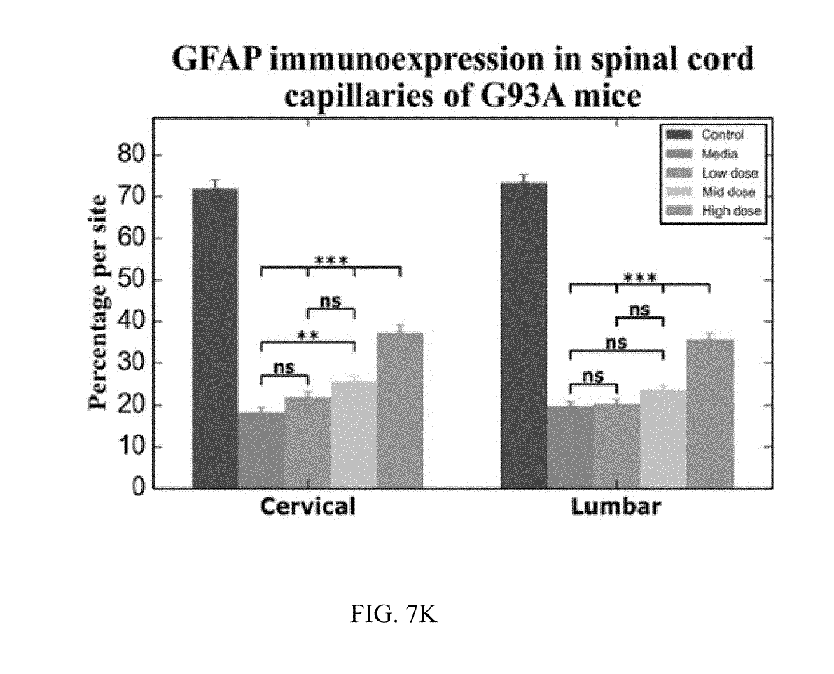

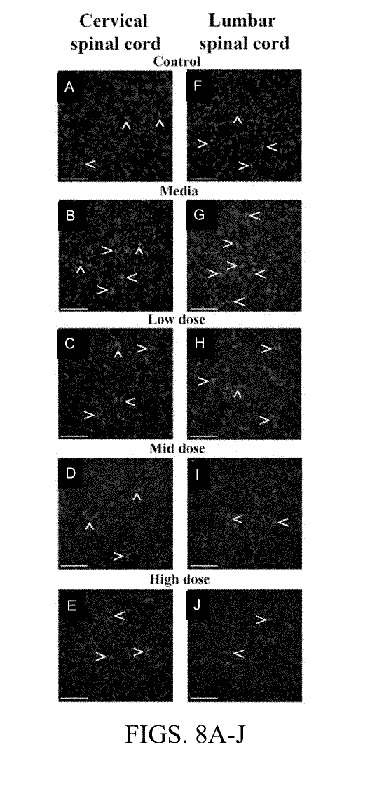

Immunohistochemical analysis of microglia in the cervical and lumbar spinal cords from cell-treated, media-injected, and control mice at 17 weeks of age was performed by fluorescent immunostaining with anti-Iba-1 antibody. In the cervical spinal cord, microglial cells with large cell bodies and thick processes at high density were determined in the ventral horn of media-injected mice (FIG. 8B) compared to control mice presenting a few microglia with fine cellular processes (FIG. 8A). In ALS mice treated with low (FIG. 8C) or mid (FIG. 8D) cell doses, a moderate decrease of Iba-1 immunoexpression was noted in the ventral horns. Fewer activated microglial cells, determined morphologically by large cell bodies and short processes, were seen in the ventral horn of the cervical spinal cords of these treated ALS mice. In mice receiving the high cell dose, more microglia with normal cellular morphology were found (FIG. 8E).

Similarly to the cervical spinal cord, microglial morphological profiles in the lumbar spinal cords from cell-treated, media-injected, and control mice at 17 weeks of age were determined. Media-treated ALS mice showed massive microgliosis in the ventral horn, as indicated by numerous microglial cells with large cell bodies and thick processes (FIG. 8G), compared to controls with typical cell morphology (FIG. 8F). Of note, more activated microglial cells were observed in the lumbar spinal cord vs. cervical in media-injected mice. In ALS mice treated with low (FIG. 8H), mid (FIG. 8I), or high (FIG. 8J) cell doses, microglial cell activation was reduced in the ventral horns. Many ramified cells with smaller cell bodies and fine cell processes at low density were detected in these animals. Particularly, mice receiving high cell dose showed Iba-1 immunoexpression in the ventral horn of the lumbar spinal cord near levels detected in control animals.

Thus, immunohistochemical analysis of Iba-1 immunoexpression in the cervical and lumbar spinal cords showed substantial microgliosis in media-injected mice at 17 weeks of age. In these mice, activated microglial cells were characterized by large cell bodies and thick processes. Four weeks after cell transplantation, microglial cell activation was significantly reduced in cell-treated mice and decreased rates of microgliosis were associated with elevated cell doses.

### Example 9

### Effect of hBM34+ Cell Transplantation at Different Doses on Motor Neuron Survival

A separate set of cervical and lumbar spinal cord sections from randomly selected cell-treated, media-injected, and control mice was stained with 0.1% cresyl violet for examination of motor neuron condition. Histological and stereological analyses of motor neurons in the ventral horn of the cervical and lumbar spinal cords were performed at 17 weeks of age.

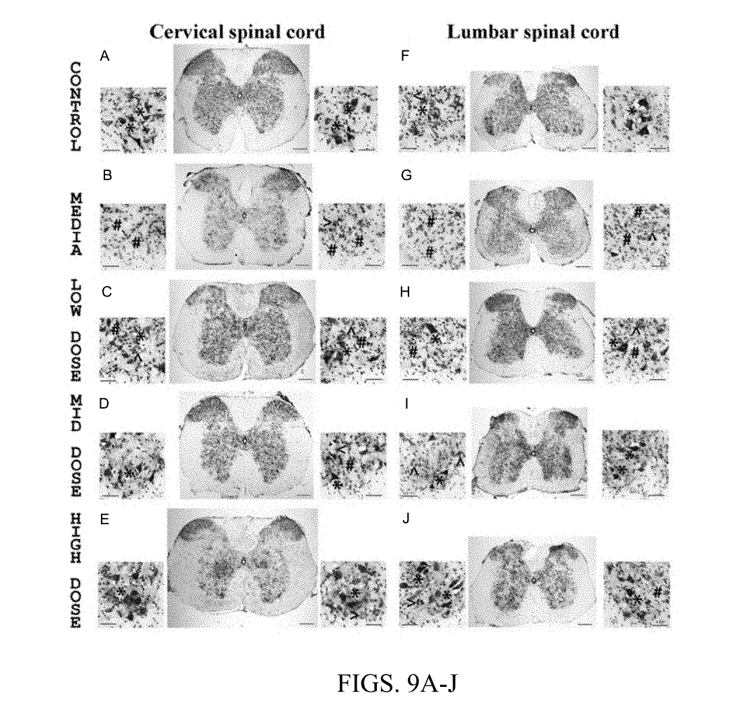

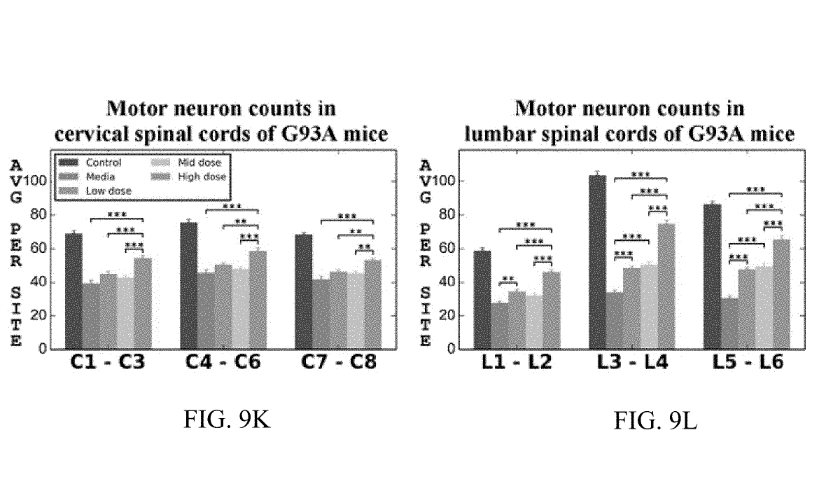

In control mice, healthy motor neurons with large soma and neuritic processes were determined in both cervical (FIG. 9A) and lumbar (FIG. 9F) spinal cords. In media-injected mice, most motor neurons had degenerated or vacuolated (FIGS. 9B,9G). Only a few healthy motor neurons were identified in the ventral horn of these mice. ALS mice receiving low (FIGS. 9C,9H) or mid (FIGS. 9D,9I) cell doses demonstrated robust appearing motor neurons in both segments of the spinal cord vs. media-injected mice. However, some degenerated motor neurons were visible. Also, differently sized motor neurons displayed vacuolization in these cell-treated mice. In contrast, many healthy motor neurons with large soma were determined in the cervical (FIG. 9E) and lumbar (FIG. 9J) spinal cords in mice receiving the high cell dose. Only a small number of motor neurons were vacuolated.

Stereological motor neuron counts were performed in discrete segmental levels in the ventral horns of cervical (C1-C3, C4-C6, and C7-C8) and lumbar (L1-L2, L3-L4, and L5-L6) spinal cords. Analysis demonstrated a significant (p<0.001) decrease of motor neuron survival in media-injected mice vs. controls at all analyzed spinal cord levels (FIGS. 9K,9L). In the cervical spinal cord, only mice receiving the high cell dose showed significantly (p<0.001 or p<0.01) higher motor neuron numbers compare to other cell-treated groups or media-injected mice (FIG. 9K). More surviving motor neurons were found at the enlarged C4-C6 cervical cord level in these animals (58.94±1.67 number/side) vs. low (50.46±1.11 number/side), mid (47.97±1.26 number/side) cell doses, or media-injected (45.67±1.84 number/side) mice.

Similarly, significantly (p<0.001) lower motor neurons counts were determined in lumbar spinal cords of media-injected mice vs. controls (FIG. 9). ALS mice receiving low dose, mid dose, or high cell doses presented significantly (at different confidence levels of p<0.001 or p<0.01) greater motor neuron survival in all discrete segmental levels compared to media-injected animals. Also, motor neuron numbers in mice receiving the high cell dose were superior (p<0.001), mainly in the enlarged lumbar level at L3-L4, compared to low or mid cell doses. Motor neuron numbers in the lumbar spinal cord at L3-L4 level in G93A mice at 17 weeks of age were: low dose—48.43±1.10, mid dose—50.42±1.87, high dose—74.84±2.10, and media—34.04±1.26 neurons/side (FIG. 9).

Thus, histological and stereological analyses of motor neuron staining for the Nissl substance in the ventral horn of the cervical and lumbar spinal cords demonstrated significant losses of motor neurons in media-injected mice at 17 weeks of age. Although some robust appearing motor neurons were indicated in spinal cords of mice with low or mid cell dose treatment, superior motor neuron survival was determined in cervical and lumbar spinal cords from mice receiving high cell dose at four weeks after transplantation.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A method of repairing vascular damage in the blood-central nervous system (CNS) barrier (B-CNS-B) of a subject comprising administering a composition comprising human bone marrow CD34+ (hBM34+) cells to a subject in need thereof, whereby the vascular damage in the B-CNS-B of the subject is repaired.

Clause 2. A method of treating amyotrophic lateral sclerosis (ALS) in a subject comprising administering a composition comprising human bone marrow CD34+ cells (hBM34+) to a subject suffering from ALS, whereby ALS is treated in the subject.

Clause 3. The method of clause 1 or 2, wherein administration of the composition comprising hBM34+ cells induces repair of vascular damage in the blood-spinal cord barrier (BSCB) of the subject.

Clause 4. The method of clause 1 or 3, wherein the vascular damage comprises endothelial cell degeneration, astrocyte end-feet alteration, tight junction protein downregulation, capillary permeability, capillary rupture, or combinations thereof.

Clause 5. The method of any one of clauses 1-4, wherein repair of vascular damage comprises endothelial cell differentiation, capillary cell engraftment, reduced capillary permeability or capillary ruptures as compared to a subject not administered the composition, re-establishment of perivascular end-feet astrocytes, or combinations thereof.

Clause 6. The method of clause 5, wherein repair of vascular damage comprises a reduction in capillary ruptures as compared to a subject not administered the composition comprising hBM34+ cells.

Clause 7. The method of any one of clauses 1-6, wherein administration of the composition comprising hBM34+ cells improves motor neuron survival as compared to a subject not administered the composition.

Clause 8. The method of any one of clauses 1-7, wherein administration of the composition comprising hBM34+ cells improves behavioral symptoms as compared to a subject not administered the composition.

Clause 9. The method of any one of clauses 1-8, wherein the subject is a mammal.

Clause 10. The method of any one of clauses 1-9, wherein the subject is a human.

Clause 11. The method of any one of clauses 1-10, wherein the composition comprising hBM34+ cells is administered intravenously.

Clause 12. The method of any one of clauses 1-12, wherein the composition comprising hBM34+ cells is administered to a jugular vein.

Clause 13. The method of any one of clauses 1-12, wherein the composition comprising hBM34+ cells is administered to the mammal once during a therapeutic period.

Clause 14. The method of any one of clauses 1-13, wherein the composition comprising hBM34+ cells is administered to the mammal two or more times during a therapeutic period.

Clause 15. The method of any one of clauses 1-14, wherein the dose of hBM34+ cells in the composition is at least about 5×10⁴, at least about 5×10⁵, or at least about 1×10⁶ of hBM34+ cells.

1. A method of repairing vascular damage in the blood-central nervous system (CNS) barrier (B-CNS-B) of a subject comprising administering a composition comprising human bone marrow CD34+ (hBM34+) cells to a subject in need thereof, whereby the vascular damage in the B-CNS-B of the subject is repaired.

2. A method of treating amyotrophic lateral sclerosis (ALS) in a subject comprising administering a composition comprising human bone marrow CD34+ cells (hBM34+) to a subject suffering from ALS, whereby ALS is treated in the subject.

3. The method of claim 1, wherein administration of the composition comprising hBM34+ cells induces repair of vascular damage in the blood-spinal cord barrier (BSCB) of the subject.

4. The method of claim 1, wherein the vascular damage comprises endothelial cell degeneration, astrocyte end-feet alteration, tight junction protein downregulation, capillary permeability, capillary rupture, or combinations thereof.

5. The method of claim 1, wherein repair of vascular damage comprises endothelial cell differentiation, capillary cell engraftment, reduced capillary permeability or capillary ruptures as compared to a subject not administered the composition, re-establishment of perivascular end-feet astrocytes, or combinations thereof.

6. The method of claim 5, wherein repair of vascular damage comprises a reduction in capillary ruptures as compared to a subject not administered the composition comprising hBM34+ cells.

7. The method of claim 1, wherein administration of the composition comprising hBM34+ cells improves motor neuron survival as compared to a subject not administered the composition.

8. The method of claim 1, wherein administration of the composition comprising hBM34+ cells improves behavioral symptoms as compared to a subject not administered the composition.

9. The method of claim 1, wherein the subject is a mammal.

10. The method of claim 1, wherein the subject is a human.

11. The method of claim 1, wherein the composition comprising hBM34+ cells is administered intravenously.

12. The method of claim 1, wherein the composition comprising hBM34+ cells is administered to a jugular vein.

13. The method of claim 1, wherein the composition comprising hBM34+ cells is administered to the mammal once during a therapeutic period.

14. The method of claim 1, wherein the composition comprising hBM34+ cells is administered to the mammal two or more times during a therapeutic period.

15. The method of claim 1, wherein the dose of hBM34+ cells in the composition is at least about 5×10⁴, at least about 5×10⁵, or at least about 1×10⁶ of hBM34+ cells.
